(19) United States
(12) Patent Application Publication
(10) Pub. No.: US 2017/0027902 A1
(43) Pub. Date: Feb. 2, 2017

(54) Use of a Composition Comprising a Flavonol, A Flavonoid, and a Fatty Acid in the Treatment of Oxidative Injuries Due to Mitochondrial Dysfunction

(71) Applicant: George Robertson, Halifax (CA)

(72) Inventors: George Robertson, Halifax (CA); Matthew Nichols, Halifax (CA)

(21) Appl. No.: 15/302,848

(22) PCT Filed: Apr. 10, 2015
(86) PCT No.: PCT/CA2015/050296

§ 371 Date: Oct. 7, 2016

Related U.S. Application Data
(60) Provisional application No. 61/978,394, filed on Apr. 11, 2014.

(51) Int. Cl. A61K 31/353; A61K 31/352; A61K 31/232

(52) U.S. Cl. CPC A61K 31/353; A61K 31/232; A61K 31/352

(57) ABSTRACT

Compositions comprising a flavan-3-ol, a flavonoid, and a fatty acid have been demonstrated to mitigate the oxidative damage arising from mitochondrial dysfunction. In preferred embodiments, the composition comprises epicatechin, quercetin, and cicosapentaenoic acid (EPA) ethyl ester. The disorders to be treated include neurodegenerative disorders such as Parkinson's disease, Huntington's disease, amyotrophic lateral sclerosis (ALS), Alzheimer's disease, and multiple sclerosis (MS); neurological damage due to stroke; and ototoxicity arising from chemotherapy with cisplatin.

### PRIOR APPLICATION INFORMATION

The instant application_claims the benefit of U.S. Provisional Patent Application, filed Apr. 11, 2014 under Ser. No. 61/978,394, entitled “Combination of Epicatechin, Quercetin and EPA ethyl ester for the prevention of hearing loss”, the contents of which are incorporated herein by reference.

### BACKGROUND OF THE INVENTION

### Mitochondrial Dysfunction is a Central Feature of Neurodegenerative Disorders

Mitochondria power cells by generating ATP. The energy required to produce ATP is created by the highly efficient transfer of electrons down a series of carriers (Complexes I-IV) that comprise the electron transport chain (ETC) [1]. This reaction is completed by the transfer of electrons to oxygen. However, if this process does not operate properly electrons leak from members of the ETC (Complexes I and III) to oxygen increasing the formation of injurious reactive oxygen species (ROS) [2-5]. The low anti-oxidant capacity and high metabolic activity of neurons render these cells particularly susceptible to ROS-mediated damage [6;7]. Oxidative injury resulting from mitochondrial dysfunction is a central pathological feature of neurodegenerative disorders such as Parkinson's disease, stroke, Huntington's disease, amyotrophic lateral sclerosis, Alzheimer's disease and multiple sclerosis [6-11]. Treatments that reduce ROS production by improving mitochondrial function have therefore attracted considerable interest as therapeutics for these disorders [9;12]. However, clinical development of neuroprotective drugs is hampered by the tremendous cost, long duration, complexity and high failure rate of human efficacy trials [13]. Identification of an acute condition resulting from pathological processes relevant to more common neurodegenerative disorders would mitigate these problems by permitting rapid proof-of-concept to be clearly established in a small group of patients.

### Cisplatin-Induced Hearing Loss

Cisplatin is a potent chemotherapeutic used to treat a wide variety of cancer types including germ cell tumors [14], nasopharyngeal carcinoma [15], lung cancer [16], ovarian cancer [17], endometrial cancer [18] and testicular cancer [19]. Unfortunately, cisplatin accumulates in the cochlea resulting in ototoxicity and hearing loss for which there is no treatment [20;21]. Ototoxicity resulting in hearing loss is also produced by other chemotherapeutics (carboplatin, oxaliplatin, vincristine), antibiotics (erythromycin, gentamicin and tobramycin), loop diuretics (furosemide), nonsteroidal anti-inflammatory drugs (aspirin, ibuprofen and naproxen) and exposure to heavy metals (mercury and lead), organic solvents (toluene, styrene or xylene) and excessive noise which is a major risk factor for presbycusis (ageing-related hearing loss). Many of the pathological processes implicated in neurodegenerative disorders including mitochondrial dysfunction leading to excessive ROS generation, calcium and iron over-loading, inflammation and apoptosis are also thought to contribute to cisplatin-induced ototoxicity [22;23]. Some audiometric studies have reported elevated hearing thresholds in 75-100% of patients treated with cisplatin that are detected within 48 hours of the end of the first course of therapy [24;25]. Assessment of drugs that may attenuate cisplatin-induced hearing loss (CIHL) can therefore be performed quickly and easily using simple audiometric tests. Children are particularly sensitive to the ototoxic effects of cisplatin, at least in part because mitochondrial enzymes such as glutathione S-transferases that combat oxidative stress are only present in the cochlea in very low amounts at this early developmental stage [26]. The high incidence of CIHL in children and recent identification of genetic markers associated with this adverse side effect in younger patients increase the likelihood that drugs which reduce CIHL will be discovered by clinical trials in this population [27].

### Flavonoids Reduce CIHL

Flavonoids are polyphenolic compounds enriched in brightly coloured fruits and vegetables that reduce the production of damaging ROS by improving mitochondrial performance [9]. Administration of Gingko biloba extract (EGb 761) to rats by intraperitoneal injection reduces CIHL [28;29]. Quercetin is one of the key components of EGb 761 responsible for the ability of this extract to reduce the ototoxic effects of cisplatin [28].

### Oxygen Glucose Deprivation (OGD) Model of Neurodegeneration

Primary cultures of rodent cortical neurons exposed to OGD model many of the essential features of the brain when blood flow is halted in ischemic stroke [30] or mitochondrial function is impaired by genetic, inflammatory or environmental factors implicated in chronic neurodegenerative disorders [31-33]. These neurons undergo rapid energetic decline, release of glutamate, failure of ATP-dependent ion pumps, generation of ROS, N-methyl-D-aspartate (NMDA) receptor hyper-stimulation, mitochondrial dysfunction, impaired autophagy and both apoptotic and necrotic cell death [34-36]. The OGD method consists of placing neurons into glucose-free medium bubbled with an anaerobic mix to remove oxygen. A sealed hypoxic chamber containing the anaerobic gas mixture is used to maintain neurons in the oxygen deprived state at physiological temperatures for various durations. By conducting experiments 10-14 days following dissection (10-14 days in vitro), excitotoxicity is developed as a consequence of neuronal expression of NMDA receptors [37]. Exposure of cortical neurons to periods of OGD of 1 hr or more are increasingly toxic with maximal cell death typically observed 24 h later [30]. Incubation of primary cultures of rodent cortical neurons with epicatechin or quercetin either before or during OGD reduces subsequent cell death [38-41]. Furthermore, it is known that some flavonoids can be toxic to the liver by inducing the expression of phase II detoxification enzymes. This potential for toxicity may be increased by combining certain flavonoids as induction of phase II enzymes elevate the metabolism and elimination of flavonoids. Furthermore, glycoside versions of flavonoids may inhibit the absorption of chemically distinct flavonoids in the gut by competing for transport by glucose carriers thus reducing their in vivo activities. Yet further, the potential benefits of combining epicatechin with quercetin against loss of neuronal viability produced by OGD have not been reported.

### SUMMARY OF THE INVENTION

According to an aspect of the invention, there is provided a method for treating, treating prophylactically, preventing or reducing the severity of oxidative injury resulting from mitochondrial dysfunction comprising administering to an individual in need of such treatment an effective amount of a composition comprising a flavan-3-ol, a flavonoid and a fatty acid.

The oxidative injury resulting from mitochondrial dysfunction may be a neurodegenerative disorder or may be ototoxicity.

The neurodegenerative disorder may be for example selected from the group consisting of Parkinson's disease; stroke; Huntington's disease; amyotrophic lateral sclerosis (ALS); Alzheimer's disease; and Multiple Sclerosis (MS).

The ototoxicity resulting in hearing loss may be produced by chemotherapeutics, such as, for example, cisplatin, carboplatin, oxaliplatin and vincristine; antibiotics, such as for example erythromycin, gentamicin and tobramycin; loop diuretics such as for example furosemide; nonsteroidal anti-inflammatory drugs such as for example aspirin, ibuprofen and naproxen; exposure to heavy metals such as for example mercury and lead; exposure to organic solvents such as for example toluene, styrene and xylene; and exposure to excessive noise which is a major risk factor for presbycusis (ageing-related hearing loss).

The individual in need of such treatment may be an individual who has been or is about to be prescribed a pharmaceutical known to or at risk of or is suspected of causing ototoxicity. An individual who is about to be prescribed such a pharmaceutical may be for example an individual who will begin taking such a pharmaceutical on a regular basis for an extended period of time starting in for example approximately one week, two weeks or one month.

Alternatively, the individual in need of such treatment may be an individual who has or is at risk of developing or who is suspected of being in early stages of Parkinson's disease; stroke; Huntington's disease; ALS; Alzheimer's disease; or MS.

According to another aspect of the invention, there is provided a composition for treating, prophylactically treating, preventing or reducing the severity of oxidative injury resulting from mitochondrial dysfunction comprising administering to an individual in need of such treatment an effective amount of a composition comprising a flavan-3-ol, a flavonoid and a fatty acid.

According to a further aspect of the invention, there is provided use of a composition comprising a flavan-3-ol, a flavonoid and a fatty acid for treating, preventing or reducing the severity of oxidative injury resulting from mitochondrial dysfunction.

According to another aspect of the invention, there is provided a method for preparing a medicament for treating, treating prophylactically, preventing or reducing the severity of oxidative injury resulting from mitochondrial dysfunction comprising admixing an effective amount of a composition comprising a flavan-3-ol, a flavonoid and a fatty acid.

According to yet another aspect of the invention, there is provided a pharmaceutical composition for treating, preventing or reducing the severity of an oxidative injury resulting from mitochondrial dysfunction comprising an effective amount of a composition comprising a flavan-3-ol, a flavonoid and a fatty acid.

According to a still further aspect of the invention, there is provided a method for treating, preventing or reducing the severity of cisplatin-induced hearing loss comprising administering to an individual in need of such treatment an effective amount of a composition comprising a flavan-3-ol, a flavonoid and a fatty acid. As will be appreciated by one of skill in the art, in this embodiment, an individual in need of such treatment is an individual who has, who is, or who is about to undergo treatment with cisplatin, as discussed herein.

### BRIEF DESCRIPTION OF THE DRAWINGS

FIG. 1. Chemical structures for the 3 compounds that comprise the composition of the invention: Epicatechin, quercetin and eicosapentaenoic acid ethyl ester.

FIG. 2. Combining quercetin (Q) and epicatechin (E) synergistically reduced the loss of cell viability for cultures of mouse primary cortical neurons exposed to oxygen glucose deprivation (OGD). A. To determine the duration of OGD that produced about a half-maximal loss of cell viability, cortical neurons were exposed to varying periods of OGD. Cell viability was assessed using the MTT assay 24 his after OGD. Three hours of OGD (180 min) produced about a 35-40% loss of cell viability while 6-9 hrs of OGD decreased viability by approximately 65% relative to control (no-OGD) cells. *p<0.05, **p<0.01, *** p<0.001 relative to control, ANOVA with Tukey's post test, mean±SEM, n=6. B. Pretreatment with Q (3 μg/ml) 24 hrs before OGD (3 hrs) modestly reduced the loss in cell viability observed 24 his after OGD. By comparison, pretreatment with Q+E (1 μg/ml) markedly protected against OGD-induced cell death. Addition of the AF4 phenolics cyanidin (C) and chlorogenic acid (D) to Q+E did not further improve the protective effects of Q+E. C. Relative to vehicle (DMSO 0.02%), pretreatment with E, but not Q, at a concentration of 1 μg/ml for 24 his modestly reduced the loss in cell viability observed 24 his after OGD (3 hrs). By comparison, pretreatment with Q+E (1 μg/ml) for 24 his synergistically decreased the injurious effects of OGD. D. Treatment with Q or E (1 μg/ml) 0.5 hrs before OGD did not reduce the loss in cell viability observed 24 hrs later. However, addition of Q+E (1 or 3 μg/ml) immediately after OGD did attenuate the injurious effects of OGD. B-D *p<0.05, **p<0.01 relative to vehicle n=3. Note that pretreatment with Q+E (1 or 3 μg/ml) for 24 hrs (C) was more effective than addition of Q+E (1 or 3 μg/ml) immediately after OGD (D). These findings suggest that quercetin and epicatechin are primarily responsible for the neuroprotective effects of AF4.

FIG. 3. Epicatechin (E) plus quercetin (Q) synergistically protect cortical neurons against lethal oxygen glucose deprivation (OGD). Detailed concentration-response relationships for neuroprotection by E, Q and E+Q against OGD were performed using the more sensitive method of Fluorescence Activated Cell Sorting (FACS) to count viable neurons (FIG. 3). Relative to control cultures (no OGD), neuronal viability was reduced to approximately 45% by only 90 min of OGD. Moreover, sub-pM concentrations of either E or Q (0.03 and 0.1 μg/ml corresponding to 0.1 and 0.3 μM) were sufficient to increase neuronal survival to 55%. By comparison, equivalent concentrations of E+Q produced much larger increases in viable neurons (˜85%). Isobolograrn analyses of the resultant concentration-cell viability response data for E, Q and E+Q generated a combination index (CI)=0.7 for E+Q (synergism: CI<1.0; performed according to Zhao et al. [42]), confirming that pretreatment with E+Q synergistically protected cortical neuron cultures against OGD. Each bar represents the mean±SEM of data from 5 experiments. *p<0.05, *p<0.05 relative to vehicle or all other groups, respectively.

FIG. 4. Addition of neuroprotective concentrations of quercetin (Q), but not epicatechin (E), to primary mouse cortical cultures increased the numbers of neurons that displayed elevated cytosolic Ca2+ concentrations [Ca2+]_{c}. Primary cultures (day 14 in vitro) were preloaded with the Ca2+ sensitive dyes fluo-3 (green; cytosolic: A-F) and x-rhod-1 (red; mitochondria: D-F) or Fluo-3 and TMRM (red; A-C) to measure the mitochondrial membrane potential. Fluorescence emissions generated by excitation of these dyes were monitored by confocal microscopy over the course of 40-60 min. A. Representative cortical culture treated with DMSO (0.02%; VEH) in which a few neurons displayed oscillations in [Ca2+]_{c} indicative of synaptic activity (thick and thin arrows, elevations of [Ca2+]_{c} that were large and small, respectively). B. Ten minutes after the addition of Q (1 μg/ml˜3 μM) to this culture there was a small increase the number of neurons that displayed oscillations in [Ca2+]_{c}. C. Ten minutes after the concentration of Q was raised to 3 μg/ml (˜10 μM) there was a further elevation in the number of neurons that displayed increased [Ca2+]_{c}. D. Representative culture treated with DMSO (0.02%; VEH). E. Ten minutes following the addition of E (3 μg/ml ˜10 μM) to this cortical culture there was no change in the number neurons that displayed elevated [Ca2+]_{c}. F. However, addition of Q (3 μg/ml) after E (3 μg/ml) did increase the number of neurons that displayed [Ca2+] _{c} oscillations ten minutes later. Scale bars=20 μm.

FIG. 5. Quercetin (0), but not epicatechin (E), increased oscillations in calcium concentrations in the cytosolic [Ca2+]_{c} and mitochondrial [Ca2+]ₘ compartments of mouse cortical neurons. A. [Ca2+]_{c} detected with fluo-3 in a representative cortical neuron (top trace) before and after the sequential additions of Q that generated concentrations of 1 and 3 μg/ml (˜3 and 10 μM) for this flavonoid in the culture dish. The mitochondrial membrane potential (red trace) monitored with TMRM was modestly elevated by concentrations of Q that increased oscillations in [Ca2+]_{c}. B. The traces shown in (A) were recorded from the neuron surrounded by the white circle. Scale bar=20 μm. C. Oscillations in [Ca2+]_{c} [fluo-3 (top trace)] and [Ca2+]ₘ [x-rhod-1 (bottom trace)] in a representative cortical neuron before and after the sequential additions of E (3 μg/ml ˜10 μM) and Q (3 μg/ml ˜10 μM). E (3 μg/ml) did not trigger either [Ca2+]_{c} (black top trace) or [Ca2+]ₘ (red bottom trace) oscillations in this neuron. However, pretreatment with E (3 μg/ml) enhanced the frequency of Q (3 μg/ml)-induced [Ca2+]_{c} oscillations (C relative to A). The induction of [Ca2+]ₘ oscillations by Q (3 μg/ml) in (C) is consistent with the ability of this flavonoid to activate the mitochondrial Ca2+ uniporter. D. The traces shown in (C) were recorded from the neuron surrounded by the white circle.

FIG. 6. Q increases spikes in cytosolic Ca²⁺ [Ca²⁺]_{c} and the mitochondrial membrane potential. Confocal imaging was used to visualize the effects of lower concentrations of Q (0.1 and 0.3 μg/ml equivalent to 0.3 and 1 μM) shown previously to be neuroprotective (see FIG. 3) on calcium dynamics and the mitochondrial membrane potential in mouse cortical neurons (FIG. 6). Primary cortical neuron cultures were co-loaded with fluo-4 to detect [Ca^{2+]}_{c} and TMRM (tetramethylrhodamine methyl ester) to measure the mitochondrial membrane potential. The sequential addition of 0.3 and 1 μM of Q produced a concentration dependent stimulation of [Ca²⁺]_{c} oscillations (bottom trace). These were accompanied by a moderate rise in basal [Ca²⁺]_{c} and elevation of the mitochondrial membrane potential (top trace), indicative of a stimulation in mitochondrial function [43]. Inhibition of Ca²⁺ recycling between mitochondria and the endoplasmic reticulum (ER) by depletion of ER Ca²⁺ stores with thapsigargin (5 μM) blocked [Ca²⁺]_{c} oscillations. The proton ionophore FCCP [carbonyl cyanide-4-(trifluoromethoxy)phenylhydrazone] that abolishes the mitochondrial membrane potential resulted in a complete loss of TMRM fluorescence, confirming specificity of this signal. The sharp increase in [Ca²⁺]_{c} in response to FCCP indicates a significant presence of Ca²⁺ in mitochondria. Increased [Ca²⁺]_{c} oscillations are directly linked to elevated mitochondrial metabolic activity by a mechanism involving the ER and mitochondrial Ca2+ uniporter [44]. Thus, our results are consistent with an increase in Ca²⁺ signaling and mitochondrial metabolism resulting from the stimulation of ER Ca²⁺ release and mitochondrial Ca2+ uniporter activity by Q.

FIG. 7. Protective concentrations of quercetin (Q) plus epicatechin (E) (1 or 3 μg/ml) elevated mitochondrial gene expression in control (no OGD) neuronal cultures and further enhanced the induction of these genes by OGD. The mRNA levels for all genes were determined by qRT-PCR and expressed relative to values obtained for control cultures that were pretreated with vehicle (VEH; DMSO 0.02%) and not exposed to OGD (relative expression). Q+E (1 and/or 3 μg/ml) elevated MT-ND2 (Complex I), SDHA (Complex II), MT-CytB (Complex Ill) and MT-ATP6 (Complex V) expression in cortical neurons not exposed to OGD 24 hrs after addition. OGD (3 hrs) elevated mRNA levels for all genes except MT-CO2 (Complex IV) 12 hrs later. Pretreatment for 24 hrs with protective concentrations of Q+E (1 or 3 μg/ml) further enhanced these OGD-induced increases with dramatic elevations occurring in the cases of MT-CO2 (8 fold) and ATP6 (14 fold). OGD also elevated PGC-1α mRNA levels that were further increased by pretreatment with Q+E (1 μg/ml). *p<0.05, **p<0.01 relative to control cultures pretreated with vehicle and not exposed OGD. p<0.05, p<0.01 relative to cortical neurons pretreated with vehicle that received OGD 24 hrs later. ANOVA with Tukey's post test, mean±SEM, n=3.

FIG. 8. Pretreatment with epicatechin (E) plus quercetin (Q) produced beneficial changes in apoptotic gene expression in cortical neurons after oxygen glucose deprivation (OGD). Levels of mRNAs are expressed relative to values for control cultures (no OGD). Consistent with the induction of p53-mediated apoptosis by OGD [45], p53 mRNA levels were elevated 12 hr after OGD relative to control cultures (no OGD). E+Q reversed these increases in a concentration-dependent manner. Neuroprotection by E+Q was also accompanied by increased mRNA levels for the anti-apoptotic gene Bcl-2. Since cAMP-response element binding protein (CREB) promotes neuronal survival by directly activating CRE elements that drive Bcl-2 expression [46-48], this observation indicates that CREB plays an important role in neuroprotection by E+Q. 0.1 μM=0.03 μg/ml; 1.0 μM=0.3 μg/ml. *p<0.05, **p<0.01 relative to vehicle. ANOVA with Tukey's post test. Bars show mean±SEM, n=3.

FIG. 9. E+Q synergistically preserved mitochondrial respiratory performance in cortical neurons exposed a lethal period of OGD. The effects of E, Q and E+Q on key aspects of mitochondrial function were assessed in cortical neuron cultures using a Seahorse Bioscience XF24 Extracellular Flux Analyzer (XF24). The XF24 creates a transient, 7 μl chamber in specialized microplates enabling oxygen concentrations associated with respiring neurons to be determined in real time. Oxygen consumption rate (OCR) is a measure of electron transport chain activity [49]. Oligomycin (ATP synthase inhibitor; 2 μM) blocks oxidative phosphorylation yielding a measure of cellular ATP production [50;51] (FIG. 6A). Relative to vehicle (0.02% DMSO), E+Q (0.1 μM) increased the suppression of OCR by oligomycin, indicating enhanced ATP production and improved mitochondrial efficiency (FIG. 6B). The remaining OCR after oligomycin treatment represents proton leak across the mitochondrial membrane [52] The reduction in proton leak produced by E+Q (0.1 μM) further indicates improved respiratory efficiency [52-54]. To determine the maximal OCR that cells can sustain, the proton ionophore (uncoupler) FCCP (2 μM) was injected. Use of FCCP at a concentration of 2 μM was established by titration studies. The difference between basal and maximal OCR is termed spare respiratory capacity (SRC) [51;55]. Relative to control neurons (FIG. 6B), maximal FCCP-induced respiration was reduced from 70% to 30% (Bottom Panel) after OGD, reflecting a loss of SRC (FIG. 6C). This loss was only partially reversed by E or Q (0.1 μM). By contrast, E+Q (0.1 μM) completely prevented SRC loss in OGD neurons. SRC confers resistance against oxidative injury (FIG. 6C) [50;56] suggesting that the preservation of SRC in OGD neurons contributes to neuroprotection by E+Q. Each point represents the mean±SEM of data from 3 experiments. *p<0.05 relative to vehicle; *p<0.05 relative to all other groups. ANOVA with Tukey's post test.

FIG. 10. CIHL was assessed by comparing auditory brain stem responses (ABRs) measured 7 days before (A) and 7 days after (B) furosemide and cisplatin. ABRs for both groups of mice measured at 4, 8, 16 and 32 kHz 7 days prior to furosemide and cisplatin were the same (A). At 7 days post furosemide and cisplatin, water-treated mice displayed greater hearing loss relative to mice treated with the composition of the invention at 8 and 16 kHz (B; *p<0.05 versus water+cisplatin, Mann-Whitney U test; each point represents the mean±SEM).

FIG. 11. Percent hair cell loss in the cochlea of mice treated with vehicle (A) or the composition of the invention (B) prior to the administration of furosemide and cisplatin. Note the loss of outer hair cells (OHC) in the vehicle treated mouse that is absent in the mouse which received the composition of the invention. A reduction of 20-30% in OHC numbers within the cochlear regions responsive to sound at 8 to 16 kHz is consistent with the observed hearing loss at these frequencies. Results are representative of 4 mice examined in the vehicle and composition groups.

FIG. 12. Oral administration of E+Q reduces brain injury in mice subjected to after an experimental stroke produced by an episode of forebrain hypoxia-ischemia (HI). Administration of either E [57] or Q [58;59] reduces ischemic brain injury, however, the effects of combining these compounds have not been reported. Two groups of adult C57BI/6 mice were dosed orally with either the composition of the invention (100 μl) or water (100 μl) once daily. Five days later, all mice were subjected to HI (40 min). Infarct volume was measured 24 hr later by staining of serial brain sections with the vital dye triphenyl tetrazolium chloride (TTC) (FIG. 12: Top Panel). Relative to vehicle-treated mice, E+Q reduced the average infarct volume from 57±6 mm³ to 33±7 mm³ (FIG. 12: Bottom Panel). This 57% reduction in infarct volume produced by E+Q is superior in magnitude to the modest 25-30% decrease in infarct volume or ischemic brain damage reported for oral administration or systemic injections of just E or Q [41;57-59]. These findings are further indicative of neuroprotective synergism produced by combining E and Q which also suggest that the composition of the invention should reduce stroke-related brain injury.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Combining quercetin with a member of the flavan-3-ol family (epicatechin), a compound known to reduce CIHL [60;61], produces synergistic increases in anti-oxidant, anti-inflammatory and neurotrophic activities [62;63]. The inventors hypothesized that combining epicatechin and quercetin would profoundly reduce CIHL. It is important to note that while there is evidence that quercetin plus epicatechin synergy may exist, there was no obvious link or clear data in the literature in support of epicatechin and quercetin synergistically protecting against cisplatin-induced hearing loss or ischemic brain damage.

To improve the oral absorption and further increase the mitochondrial trophic properties of these 2 lipophilic compounds, the inventors administered them in eicosapentaenoic acid (EPA) ethyl ester. EPA ethyl ester enhances mitochondrial biogenesis [64], protects against cisplatin-induced neurotoxicity [65], decreases the risk of ageing-related hearing loss [66] and when combined with quercetin or epicatechin markedly increases the oral bioavailability and therapeutic effects of these flavonoids in rodent models of Huntington's disease and Alzheimer's disease [67;68]. Since mitochondrial dysfunction is a common pathological mechanism in CIHL and chronic neurodegenerative disorders such as Parkinson's disease, stroke, multiple sclerosis and Alzheimer's disease [9], efficacy against CIHL in humans would also strongly suggest effectiveness against these disorders.

According to an aspect of the invention, there is provided a method for preventing, reducing the severity of, treating prophylactically or treating oxidative injury resulting from mitochondrial dysfunction comprising administering to an individual in need of such treatment an effective amount of a composition comprising a flavan-3-ol, a flavonoid and a fatty acid.

The oxidative injury resulting from mitochondrial dysfunction may be a neurodegenerative disorder or may be ototoxicity.

The neurodegenerative disorder may be for example selected from the group consisting of Parkinson's disease; stroke; Huntington's disease; amyotrophic lateral sclerosis (ALS); Alzheimer's disease; and Multiple Sclerosis (MS).

The ototoxicity resulting in hearing loss may be produced by chemotherapeutics, such as, for example, cisplatin, carboplatin, oxaliplatin and vincristine; antibiotics, such as for example erythromycin, gentamicin and tobramycin; loop diuretics such as for example furosemide; nonsteroidal anti-inflammatory drugs such as for example aspirin, ibuprofen and naproxen; exposure to heavy metals such as for example mercury and lead; exposure to organic solvents such as for example toluene, styrene and xylene; and/or exposure to excessive noise which is a major risk factor for presbycusis (ageing-related hearing loss).

The individual in need of such treatment may be an individual who has been or is about to be prescribed a pharmaceutical known to or at risk or is suspected of causing ototoxicity. An individual who is about to be prescribed such a pharmaceutical may be for example an individual who is will begin taking such a pharmaceutical on a regular basis for an extended period of time starting in for example approximately one week, two weeks or one month.

Alternatively, the individual in need of such treatment may be an individual who has or is at risk of developing or who is suspected of being in early stages of or who has a familial history of or genetic predisposition for: Parkinson's disease; stroke; Huntington's disease; ALS; Alzheimer's disease; or MS.

According to a further aspect of the invention, there is provided use of a composition comprising a flavan-3-ol, a flavonoid and a fatty acid for treating, preventing or reducing the severity of oxidative injury resulting from mitochondrial dysfunction.

According to another aspect of the invention, there is provided a method for preparing a medicament for treating, treating prophylactically, preventing or reducing the severity of oxidative injury resulting from mitochondrial dysfunction comprising admixing an effective amount of a composition comprising a flavan-3-ol, a flavonoid and a fatty acid.

According to yet another aspect of the invention, there is provided a pharmaceutical composition for treating, treating prophylactically, preventing or reducing the severity of an oxidative injury resulting from mitochondrial dysfunction comprising an effective amount of a composition comprising a flavan-3-ol, a flavonoid and a fatty acid.

In a preferred embodiment, the composition or pharmaceutical composition comprises 10-250 mg flavan-3-ol and 10-250 mg flavonoid suspended in 0.1-1 ml of a suitable fatty acid.

In a preferred embodiment, the composition or pharmaceutical composition comprises epicatechin, a flavonoid and a suitable fatty acid, for example, 10-250 mg epicatechin and 10-250 mg flavonoid suspended in 0.1-1 ml of a suitable fatty acid.

In a preferred embodiment, the composition or pharmaceutical composition comprises a flavan-3-ol, quercetin and a suitable fatty acid, for example, 10-250 mg flavan-3-ol and 10-250 mg quercetin suspended in 0.1-1 ml of a suitable fatty acid.

In a preferred embodiment, the composition or pharmaceutical composition comprises epicatechin, quercetin and a suitable fatty acid, 10-250 mg epicatechin and 10-250 mg quercetin suspended in 0.1-1 ml of a suitable fatty acid.

In a preferred embodiment, the composition or pharmaceutical composition comprises epicatechin, quercetin and eicosapentaenoic acid ethyl ester, for example, 10-250 mg epicatechin and 10-250 mg quercetin suspended in 0.1-1 ml of eicosapentaenoic acid ethyl ester. Preferably, the pharmaceutical composition is formulated for oral administration.

In preferred embodiments, the flavonoid is selected from the group consisting of Quercetin; Isorhamnetin (3-Methylquercetin, 3,5,7-trihydroxy-2-(4-hydroxy-3-methoxyphenyl)chromen-4-one); Rutin (Quercetin-3-O-rutinoside, 2-(3,4-dihydroxyphenyl)-5,7-dihydroxy-3-[α-L-rhamnopyranosyl-(1→6)-β-D-glucopyranosyloxy]-4H-chromen-4-one); Quercitrin (Quercetin 3-O-rhamnoside, 2-(3,4-Dihydroxyphenyl)-5,7-dihydroxy-3-[[(2S,3R,4R,5R,6S)-3,4,5-trihydroxy-6-methyl-2-tetrahydropyranyl]oxy]-4-chromenone); Hyperoside (Quercetin-3-O-galactoside, 2-(3,4-dihydroxyphenyl)-3-[(3R,4S,5R,6R)-3,4,5-trihydroxy-6-(hydroxymethyl)oxan-2-yl]oxy-4H-chromene-4,5,7-triol); Taxifolin (Dihydroquercetin, (2R,3R)-2-(3,4-dihydroxyphenyl)-3,5,7-trihydroxy-2, 3-dihydrochromen-4-one); Querectin-3-O-glucoside; Quercetin-3-O-maltoside; Quercetin-3-O-gentiobioside; α-monoglucosyl rutin; α-oligoglucosyl rutin; α-oligogiucosyl isoquercitrin (enzymatically modified isoquercitrin, EMIQ); Apigenin (5,7-Dihydroxy-2-(4-hydroxyphenyl)-4H-1-benzopyran-4-one); Homoeriodictyol ((2S)-5,7-Dihydroxy-2-(4-hydroxy-3-methoxyphenyl)-4-chrorrianone); Galangin (3,5,7-trihydroxy-2-phenylchromen-4-one); Myricetin (3,5,7-Trihydroxy-2-(3,4,5-trihydroxyphenyl)-4-chromenone; Kaempferol (3,5,7-Trihydroxy-2-(4-hydroxyphenyl)-4H-chromen-4-one); Aromadendrin (Dihydrokaempferol, (2R, 3R)-3,5,7-trihydroxy-2-(4-hydroxyphenyl)-2,3-dihydrochromen-4-one); Pachypodol (5-hydroxy-2-(4-hydroxy-3-methoxyphenyl)-3,7-dimethoxychromen-4-one); Fisetin (2-(3,4-dihydroxyphenyl)-3,7-dihydroxychromen-4-one); Hesperetin ((S)-2,3-dihydro-5,7-dihydroxy-2-(3-hydroxy-4-methoxyphenyl)-4H-1-benzopyran-4-one); Hesperidin (Hesperetin 7-rutinoside); Baicalein (5,6,7-Trihydroxy-2-phenyl-chromen-4-one); Baicalin (Baicalein 7-O-glucuronide); Tetuin (Baicalein 6-O-glucoside); Luteolin (2-(3,4-Dihydroxyphenyl)-5,7-dihydroxy-4-chromenone); Cynaroside (Luteolin 7-O-glucoside); Isoorientin (Luteolin 6-C-glucoside); Orientin (Luteolin 8-C-glucoside); Veronicastroside (Luteolin 7-O-neohesperidoside); Luteolin-7-O-glucuronide; Tangeritin (Tangeretin, 5,6,7,8-tetramethoxy-2-(4-methoxyphenyl)-4H-1-benzopyran-4-one); Giraldiin A (4′,5,5′,7-Tetrahydroxy-3-methoxy-3′-O-α-L-arabinopyranosyloxyflavone); Giraldiin B (5,5′,7′-Trihydroxy-2′,3-dimethoxy-4′-O-β-D-glucopyranosyloxyflavone); Naringenin (5,7-dihydroxy-2-(4-hydroxyphenyl)chroman-4-one); Nepetin (2-(3,4-dihydroxyphenyl)-5,7-dihydroxy-6-methoxychronnen-4-one); Nepitrin (Nepetin 7-O-glucoside); Rhamnazin (3,5-dihydroxy-2-(4-hydroxy-3-methoxyphenyl)-7-methoxychromen-4-one) Wogonin (5,7-Dihydroxy-8-methoxy-2-phenyl-4H-chromen-4-one); and Scutellarin (7-(β-D-glucopyranuronosyloxy)-5,6-dihydroxy-2-(4-hydroxyphenyl)-4H-1-benzopyran-4-one).

In preferred embodiments, the flavan-3-ol is selected from the group consisting of: (−)-Epicatechin; (+)-Catechin ((2R,3S)-2-(3,4-dihydroxyphenyl)-3,4-dihydro-2H-chromene-3,5,7-triol); (−)-Catechin ((2R,3R)-2-(3,4-dihydroxyphenyl)-3,4-dihydro-2H-chromene-3,5,7-triol); (+)-Epicatechin (2S,3S) ((2S,35)-2-(3,4-dihydroxyphenyl)-3,4-dihydro-2H-chromene-3,5,7-triol); Gallocatechin ((2R,3R)-2-(3,4,5-trihydroxyphenyl)-3,4-dihydro-2H-1-benzopyran-3,5,7-triol); and Epigallocatechin gallate (EGCG) [(2R,3R)-5,7-dihydroxy-2-(3,4,5-trihydroxyphenyl)chroman-3-yl] 3,4,5-trihydroxybenzoate.

In preferred embodiments, the fatty acid is selected from the group consisting of: EPA ethyl ester; Eicosapentaenoic acid (EPA (5Z,8Z,11Z, 14Z, 17Z)-5,8,11,14,17-icosapentae noic acid); Docosahexaenoic acid ((4Z,7Z,10Z,13Z,16Z,19Z)-docosa-4,7,10,13,16,19-hexaenoic acid); α-Linolenic acid ((9Z,12Z,15Z)-9,12,15-Octadecatrienoic acid); γ-Linolenic acid (all-cis-6,9,12-octadecatrienoic acid); Linoleic acid ((9Z,12Z)-9,12-Octadecadienoic acid); Lipoic acid ((R)-5-(1,2-dithiolan-3-yl)pentanoic acid); Oleic acid ((9Z)-Octadec-9-enoic acid); Palmitoleic acid (hexadec-9-enoic acid); Vaccenic acid ((E)-Octadec-11-enoic acid); Rumenic acid ((9Z,11E)-Octadeca-9,11-dienoic acid); Hexadecatrienoic acid (HTA) (all-cis 7,10,13-hexadecatrienoic acid); Stearidonic acid (SDA) (all-cis-6,9,12,15,-octadecatetraenoic acid); Eicosatrienoic acid (ETE) (all-cis-11,14,17-eicosatrienoic acid); vitamin E (α-tocopherol, γ-tocopherol); icosatetraenoic acid (ETA) (all-cis-8,11,14,17-eicosatetraenoic acid); Heneicosapentaenoic acid (HPA) (all-cis-6,9,12,15,18-heneicosapentaenoic acid); Docosapentaenoic acid (DPA, Clupanodonic acid (all-cis-7,10,13,16,19-docosapentaenoic acid)); Tetracosapentaenoic acid (all-cis-9,12,15,18,21-tetracosapentaenoic acid); Tetracosahexaenoic acid (Nisinic acid (all-cis-6,9,12,15,18,21-tetracosahexaenoic acid)); Eicosadienoic acid (all-cis-11,14-eicosadienoic acid); Dihomo-gamma-linolenic acid (DGLA (all-cis-8,11,14-eicosatrienoic acid)); Arachidonic acid (all-cis-5,8,11,14-eicosatetraenoic acid); Docosadienoic acid (all-cis-13,16-docosadienoic acid); Adrenic acid (all-cis-7,10,13,16-docosatetraenoic acid); Docosapentaenoic acid (Osbond acid (all-cis-4,7,10,13,16-docosapentaenoic acid)); Tetracosatetraenoic acid (all-cis-9,12,15,18-tetracosatetraenoic acid); Tetracosapentaenoic acid (all-cis-6,9,12,15,18-tetracosapentaenoic acid); Eicosenoic acid (cis-11-eicosenoic acid); Mead acid (all-cis-5,8,11-eicosatrienoic acid); Erucic acid (cis-13-docosenoic acid); Nervonic acid (cis-15-tetracosenoic acid); α-Calendic acid (8E,10E,12Z-octadecatrienoic acid); β-Calendic acid (8E,10E,12E-octadecatrienoic acid); Jacaric acid (8Z,10E,12Z-octadecatrienoic acid); α-Eleostearic acid (9Z,11E,13E-octadeca-9,11,13-trienoic acid); β-Eleostearic acid (9E,11E,13E-octadeca-9,11,13-trienoic acid); Catalpic acid (9Z,11Z,13E-octadeca-9,11,13-trienoic acid); Punicic acid (9Z,11E,13Z-octadeca-9,11,13-trienoic acid); Rumelenic acid (9E,11Z,15E-octadeca-9,11,15-trienoic acid); α-Parinaric acid (9E,11Z,13Z,15E-octadeca-9,11,13,15-tetraenoic acid); β-Parinaric acid (all trans-octadeca-9,11,13,15-tretraenoic acid); Bosseopentaenoic acid (5Z,8Z,10E,12E,14Z-eicosanoic acid); Pinolenic acid ((5Z,9Z,12Z)-octadeca-5,9,12-trienoic acid); Podocarpic acid ((5Z,11Z,14Z)-eicosa-5,11,14-trienoic acid) and plasmalogens which are glycerophospholipids containing a fatty alcohol linked by a vinyl ether bond to the sn-1 position of the glycerol backbone and enriched in DHA and arachidonic acid (AA) at the sn-2 position. An example of suitable plasmalogens would be phosphatidylethanolamine plasmalogens.

As discussed below, at concentrations of 10 μg/ml or above quercetin or epicatechin or epicatechin plus quercetin were increasingly toxic to primary cultures of mouse cortical neurons. Thus, the therapeutic range is therefore 0.03-3 μg per ml or 0.1-10 μM.

According to a still further aspect of the invention, there is provided a method for treating, treating prophylactically, preventing or reducing the severity of cisplatin-induced hearing loss comprising administering to an individual in need of such treatment an effective amount of a composition comprising a flavan-3-ol, a flavonoid and a fatty acid. As will be appreciated by one of skill in the art, in this embodiment, an individual in need of such treatment is an individual who has, who is, or who is about to undergo treatment with cisplatin, as discussed herein.

In some embodiments, the flavan-3-ol is (−)-Epicatechin, the flavonoid is Quercetin and the fatty acid is EPA ethyl ester, as discussed herein.

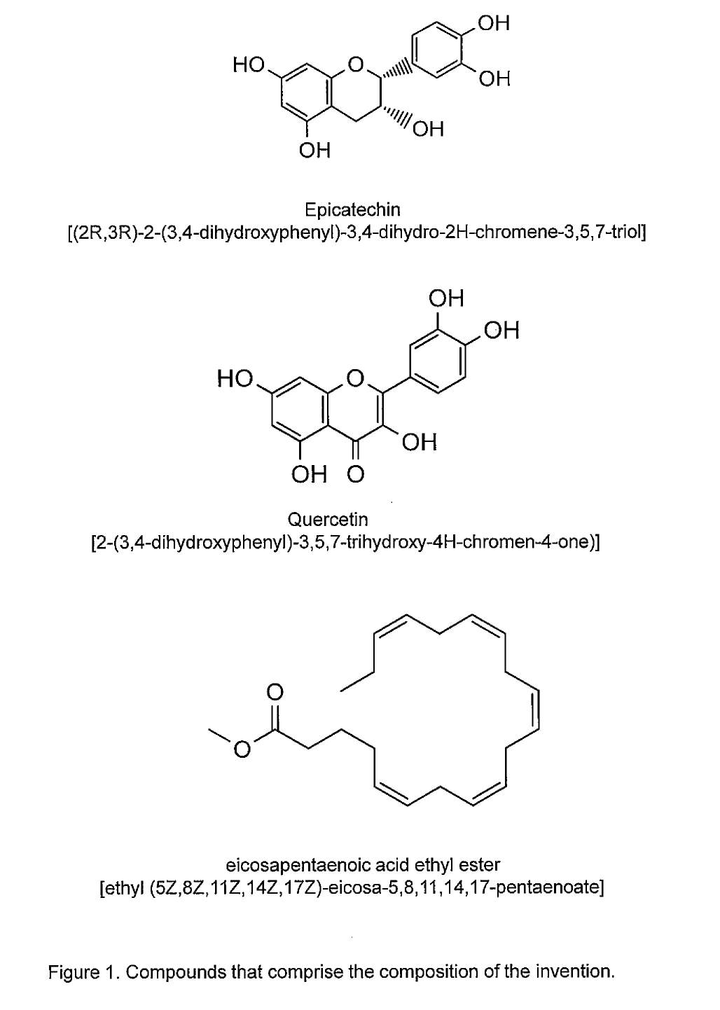

Described herein is a combination of three natural compounds: Epicatechin [E; (2R,3R)-2-(3,4-dihydroxyphenyl)-3,4-dihydro-2H-chromene-3,5,7-triol] and quercetin [QU; 2-(3,4-dihydroxyphenyl)-3,5,7-trihydroxy-4H-chromen-4-one)] suspended in eicosapentaenoic acid ethyl ester [EPA; ethyl (5Z,8Z,11Z,14Z,17Z)-eicosa-5,8,11,14,17-pentaenoate] (FIG. 1). Epicatechin (3.0 mg) plus quercetin (3.0 mg) are mixed with 1 g of eicosapentaenoic acid ethyl ester to generate 1 ml of the composition of the invention. Epicatechin and quercetin are flavonoids abundant in apples, dark chocolate and green tea. Eicosapentaenoic acid ethyl ester (EPA) is a chemically modified polyunsaturated fatty acid obtained from fish oil. The therapeutic potential of these compounds is supported by their safety [69;70], efficacy in pre-clinical models for a wide variety of neurodegenerative disorders [9;71] and evidence that habitual consumption of dietary sources rich in flavonoids or EPA reduces the risk for Parkinson's disease (PD) [72;73], stroke [74;75] and Alzheimer's disease (AD) [76;77]. Combining either quercetin or epicatechin with EPA markedly enhances their oral absorption and therapeutic effects in rodent models for Huntington's disease and Alzheimer's disease [67;68].

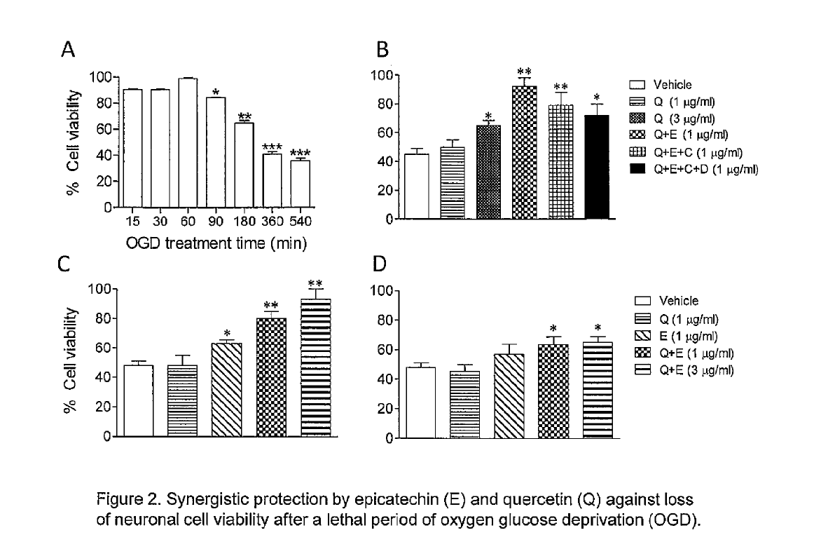

It is noted that as shown in FIG. 2, adding cyanidin and chlorogenic acid to quercetin plus epicatechin added no further protective benefits. While not wishing to be bound to a particular theory or hypothesis, the inventors believe that compounds belonging these chemical classes (anthocyanidins, anthocyanins, 3-hydroxyanthocyanidins, 3-deoxyanthocyanidins, 3-hydroxyanthocyanidins, hydroxycinnamic acids, caffeic acid, ferulic acid, p-coumaric acid) should not have therapeutic value against cisplatin-induced hearing loss when combined with epicatechin and quercetin.

Quercetin and epicatechin are dietary flavonoids with well established efficacy in a wide variety of in vitro and in vivo models for neurodegenerative disorders. As discussed herein, combining quercetin with epicatechin synergistically improved the survival of primary cultures of mouse cortical neurons subjected to a lethal period of oxygen glucose deprivation (OGD). Cortical neurons treated with quercetin and with epicatechin displayed supra-additive increases in oscillations of intracellular and mitochondrial calcium concentrations indicative of increased synaptic activity. These increases were accompanied by dramatic elevations in mRNA levels for genes encoding members of Complexes I-V of the respiratory chain implicating improved mitochondrial performance in the neuroprotective effects of these compounds. Epicatechin and quercetin also increased neuronal mitochondrial ATP production and reduced proton leak in a synergistic manner. Combining epicatechin with quercetin completely prevented the loss of protective spare respiratory capacity in cortical neurons subjected to a lethal period of oxygen-glucose deprivation. These findings further support the ability of epicatechin and quercetin synergistically protect neurons against oxidative stress by markedly enhancing mitochondrial performance. Epicatechin and quercetin have been previously hypothesized to protect cancer patients from cisplatin-induced hearing loss by enhancing mitochondrial function in vulnerable sensory hair cells of the cochlea. It is important to note that while there is evidence that quercetin plus epicatechin synergy may exist, there was no obvious link or clear data in the literature in support of epicatechin and quercetin synergistically protecting against cisplatin-induced hearing loss.

To improve the oral absorption of epicatechin (E; 3 mg) and quercetin (QU; 3 mg) these lipophilic compounds were dissolved in eicosapentaenoic acid (EPA) ethyl ester (1 ml) to generate a novel therapeutic composition. Pre-dosing mice orally with the composition (100 μl; once daily) beginning 7 days before intraperitoneal injections of furosemide (200 mg/kg) and cisplatin (0.2 mg/kg) reduced the death of sensory hair cells in the cochlea and hearing loss detected 7 days later. The large increases in mitochondrial calcium transients, gene expression and respiratory performance produced by combining epicatechin and quercetin suggest that the composition of the invention protected against furosemide and cisplatin-induced hearing loss by improving mitochondrial performance in vulnerable sensory hair cells.

Pre-dosing mice orally with the composition of the invention reduced brain damage in a model of hypoxic-ischemic brain injury that mimics the unfavourable conditions responsible for stroke. Cortical neurons pretreated with epicatechin and quercetin were protected against loss of mitochondrial respiratory function after injurious oxygen-glucose deprivation. Mitochondrial dysfunction has been implicated in stroke brain injury [14-16] indicating that the composition of the invention will reduce the risk of brain damage after a stroke by preserving mitochondrial function.

Supra-Additive Neuroprotection Produced by Combining Epicatechin with Quercetin

A major finding of this study was that combining epicatechin with quercetin synergistically reduced the loss of cell viability for cortical neurons subjected to a lethal period of OGD. Epicatechin produce a modest (30%) increase in neuronal survival at a concentration of 1 μg/ml (˜3 μM) while quercetin failed to protect cortical neurons against OGD at this concentration. When epicatechin and quercetin were combined at a concentration of 0.5 μg/ml each to achieve a total flavonoid concentration of 1 μg/ml these compounds generated a 65% increase in neuronal survival. At a concentration of 3 μg/ml (˜10 μM) quercetin increased cortical neuron viability after OGD by 30%, however, combining just half this concentration of quercetin (1.5 μg/ml ˜5 μM) with epicatechin (1.5 μg/ml ˜5 μM) increased neuronal survival to 85%. Detailed concentration-response relationships for neuroprotection by E, Q and E+Q against OGD performed using the more sensitive method of Fluorescence Activated Cell Sorting (FACS) to count viable neurons confirmed synergistic neuroprotection by epicatechin plus quercetin. Relative to control cultures (no OGD), neuronal viability was reduced to approximately 45% by only 90 min of OGD. Moreover, sub-pM concentrations of either E or Q (0.03 or 0.1 μg/ml; 0.1 or 0.3 μM) were sufficient to increase neuronal survival to 55%. By comparison, equivalent concentrations of E+Q produced much larger increases in viable neurons (˜85%). lsobologram analyses of the resultant concentration-cell viability response data for E, Q and E+Q generated a combination index (CI)=0.7 for E+Q (synergism: CI<1.0), confirming that pretreatment with E+Q synergistically protected cortical neuron cultures against OGD. Although these compounds have been shown to protect primary cultures of rodent cortical neurons against OGD-induced cell death [39;41], we are not aware of a previous report demonstrating that combining epicatechin with quercetin produces synergistic increases in neuroprotection.

### Epicatechin Enhances Quercetin-Induced Oscillations in Cytosolic and Mitochondrial Calcium Concentrations

Cortical neurons grown in primary culture develop extensive processes and form functional synaptic connections, resulting in spontaneous oscillations in intracellular calcium concentrations [Ca²⁺]_{c} or calcium transients indicative of neuronal activity [78;79]. Addition of quercetin to cortical neuron cultures increased the number of neurons that displayed calcium transients and the frequency of these events. Treatments that increase synaptic activity such as NMDA receptor agonists or γ-aminobutyric acid receptor antagonists activate signaling events which increase the resistance of cortical neurons to excitotoxic/ischemic injury [80-82]. This suggests that similar mechanisms are responsible for the protective effects of quercetin against OGD. Although epicatechin failed to increase calcium transients, it did enhance the ability of quercetin to increase [Ca²⁺]_{c} oscillations, further implicating elevated synaptic activity in the neuroprotective mechanisms responsible for the synergistic benefits of these compounds in the OGD model. Quercetin has been reported to increase mitochondrial calcium concentration by directly activating the mitochondrial calcium uniporter (MCU) [83]. In keeping with this finding, addition of quercetin after epicatechin increased [Ca²⁺]ₘ oscillations in cortical neurons. Calcium entry into mitochondria stimulates respiratory capacity by activity of mitochondrial enzymes necessary for generating reducing equivalents [84], metabolic substrates [85] and electron transfer [86]. Consistent with these findings, quercetin increased the mitochondrial membrane potential indicative of enhanced cellular bioeneregetics [44]. Compounds that improve respiratory capacity are neuroprotective in genetic models of Huntington's disease [87] and Parkinson's disease [88]. Combining epicatechin with quercetin has resulted in cortical neurons protected against OGD-induced cell death by synergistically increasing calcium-mediated elevations in respiratory capacity.

### Epicatechin Plus Quercetin Synergistically Improve Key Aspects of Mitochondrial Performance

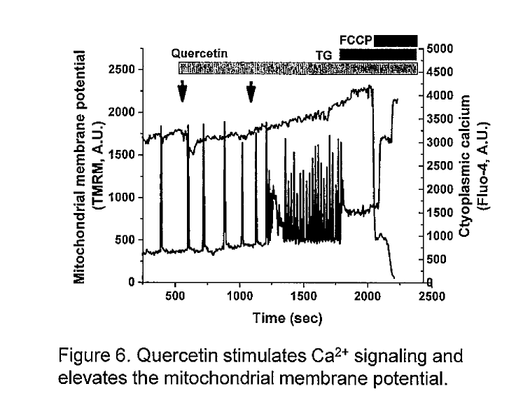

E+Q synergistically preserve mitochondrial respiratory performance in cortical neurons exposed to a lethal period of OGD. The effects of E, Q and E+Q on key aspects of mitochondrial function were assessed in cortical neuron cultures using a Seahorse Bioscience XF24 Extracellular Flux Analyzer (XF24). The XF24 creates a transient, 7 μl chamber in specialized microplates enabling oxygen concentrations associated with respiring neurons to be determined in real time. Oxygen consumption rate (OCR) is a measure of electron transport chain activity [49]. Oligomycin (ATP synthase inhibitor; 2 μM) blocks oxidative phosphorylation yielding a measure of cellular ATP production [50;51] (FIG. 6A). Relative to vehicle (0.02% DMSO), E+Q (0.1 μM) increased the suppression of OCR by oligomycin, indicating enhanced ATP production and improved mitochondrial efficiency (FIG. 6B). The remaining OCR after oligomycin treatment represents proton leak across the mitochondrial membrane [52]. The reduction in proton leak produced by E+Q (0.1 μM) further indicates improved respiratory efficiency [52;53]. To determine the maximal OCR that cells can sustain, the proton ionophore (uncoupler) FCCP (2 μM) was injected. Use of FCCP at a concentration of 2 μM was established by titration studies. The difference between basal and maximal OCR is termed spare respiratory capacity (SRC) [55]. SRC enables neurons to cope with the added energetic demands imposed by neurotransmission [51]. Relative to control neurons (FIG. 6B), maximal FCCP-induced respiration was reduced from 70% to 30% (Bottom Panel; black line) after OGD, reflecting a loss of SRC (FIG. 6C). This loss was only partially reversed by E or Q (0.1 μM). By contrast, E+Q (0.1 μM) completely prevented SRC loss in OGD neurons. SRC confers resistance against oxidative injury (FIG. 6C), [50;56] suggesting that the preservation of SRC in OGD neurons contributes to neuroprotection by E+Q.

### Epicatechin and Quercetin Increases ETC and Pro-Survival Gene Expression by OGD

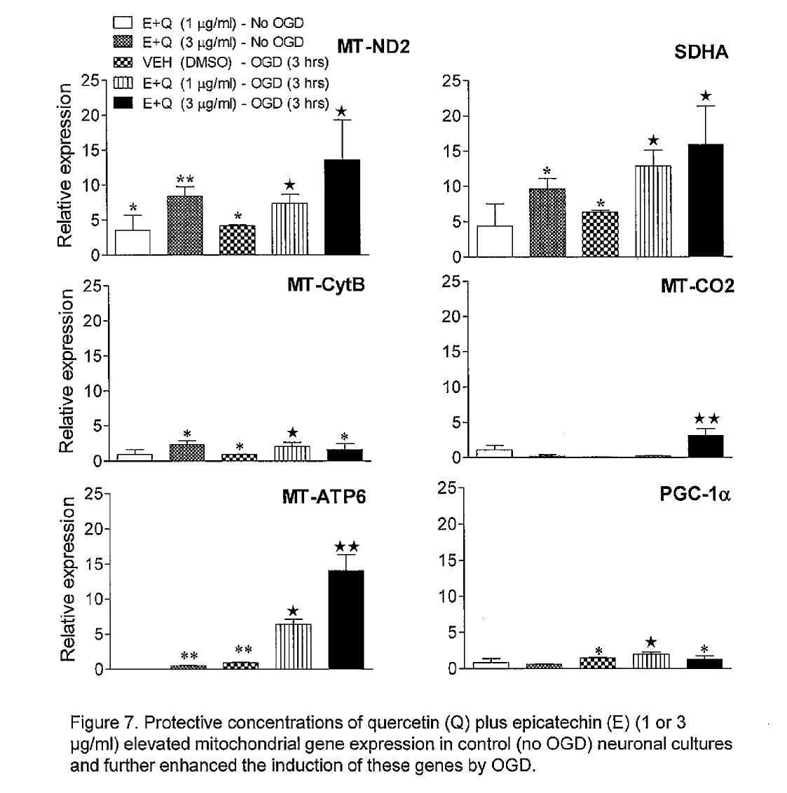

Consistent with a proposed increase in respiratory capacity, epicatechin plus quercetin (1 or 3 μg/ml 3 or 10 μM) elevated mRNAs levels for several mitochondrial (MT) and nuclear genes that encode members of Complex I (MT-ND2), Complex II (SDHA, nuclear encoded), Complex III (MT-CytB) and Complex V (MT-ATP6). Furthermore, pretreatment with epicatechin plus quercetin (1 or 3 μg/ml ˜3 or 10 μM) markedly enhanced the induction by OGD of these genes as well as MT-CO2 in a concentration-dependent manner (FIG. 7). Mitochondria possess an endogenous cyclic adenosine monophosphate (cAMP) signaling system [89;90] that is activated by calcium entry into this organelle [91] resulting in the elevated expression and enzymatic activity of several respiratory gene products [54;89]. Quercetin [92] and epigallocatechin-3-gallate [93], a flavonoid structurally similar to epicatechin, rapidly accumulate in mitochondria where these compounds may raise cAMP levels by blocking phosphodiesterase activity [94-96] intrinsic to this organelle [97]. In the presence of this flavonoid-mediated inhibition of phosphodiesterase activity, stimulation of adenylate cyclase by quercetin-induced mitochondrial calcium uptake would be expected to result in much larger increases in cAMP levels. This is supported by the enhanced induction of Bcl-2, an anti-apoptotic gene whose expression is driven by CREB, in neurons pretreated with protective concentrations of epicatechin and quercetin after oxygen-glucose deprivation [46;48]. In these ways, combining epicatechin with quercetin produces synergistic increases in mitochondrial performance and pro-survival gene expression that confer marked resistance to oxidative damage [54;98-101].

### Combination Treatment Reduces Cisplatin-Induced Ototoxicity and Hearing Loss

Following systemic administration of cisplatin, this compound is transported across the bloodendolymph barrier in the cochlea [102] and becomes preferentially concentrated by the copper transporter (CTR1) in sensory hair cells [103]. Cisplatin is then transported into mitochondria of sensory hair cells by the copper chaperone Cox17 [104]. Once inside the mitochondrion, cisplatin binds with high affinity to DNA, inhibiting both transcription and DNA repair that blocks the synthesis of respiratory proteins, resulting in excessive ROS production, sensory hair cell death and hearing loss [105;106]. Intratympanic injection of epicatechin which generates high concentrations of this flavonoid in the cochlea protect rats against cisplatin-induced sensory hair loss [60]. Administration of Gingko biloba extract (EGb 761) which contains quercetin by intraperitoneal injection also protects rats against cisplatin-induced ototoxicity [28;29]. However, the potential benefits of combined administration of epicatechin and quercetin against cisplatin-induced ototoxicity and hearing loss have not yet been reported. Epicatechin was combined with quercetin to create a formulation that incorporated the protective benefits of these two chemically distinct compounds. In order to improve the oral bioavailability of epicatechin and quercetin these compounds were dissolved in EPA ethyl ester to generate a novel therapeutic formulation. Since long-term assessment of cisplatin-induced hearing loss is prevented by mortality due to renal toxicity, furosemide was administered 1 hr before cisplatin to protect the kidneys from damage by this chemotherapeutic [107]. Oral administration of the composition beginning 7 days before injections of furosemide and cisplatin reduced hearing loss and sensory hair cell death assessed 7 days later. The profound ability of epicatechin plus quercetin to protect cortical neurons against OGD-induced cell death by improving mitochondrial function suggests that similar mechanisms are responsible for the prevention of cisplatin-induced ototoxicity by the composition of the invention. EPA ethyl ester which has been shown to reduce cisplatin-induced neurotoxicity [65] and decrease the risk of ageing-related hearing loss [66] may also have enhanced the protective effects of epicatechin and quercetin by increasing mitochondrial biogenesis [64].

Mitochondrial dysfunction is a central feature of most neurodegenerative disorders indicating that compounds which improve the performance of this organelle will be effective treatments for these conditions. Habitual consumption of a diet enriched in flavonoids that reduce ROS production by enhancing mitochondrial function decrease the risk of Parkinson's disease, stroke and dementia [72;74;76]. Combining epicatechin with quercetin synergistically reduced the loss of neuronal viability after OGD in a fashion that was closely associated with improved mitochondrial performance. Concordant increases in mitochondrial calcium uptake and cAMP-mediated signaling resulting in supra-additive increases in the expression and activity of enzymes that comprise the ETC are proposed to mediate the synergistic effects of these compounds on mitochondrial performance and neuronal survival. Oral administration of epicatechin and quercetin dissolved in EPA ethyl ester protected mice against cisplatin-induced ototoxicity and hearing loss. Since cisplatin-induced hearing loss occurs within days of the first cycle of chemotherapy, clinical trials designed to assess the protective efficacy of the composition could be completed rapid using simple audiometric tests. Furthermore, cancer patients selected for chemotherapy may be pretreated with the composition of the invention prior to the onset chemotherapy. This would maximise the potential for therapeutic efficacy by permitting adequate time for transcriptional events to occur that mediate improved mitochondrial performance.

### Results

Combining Epicatechin with Quercetin Synergistically Protected Primary Cultures of Mouse Cortical Neurons against OGD-Induced Cell Death

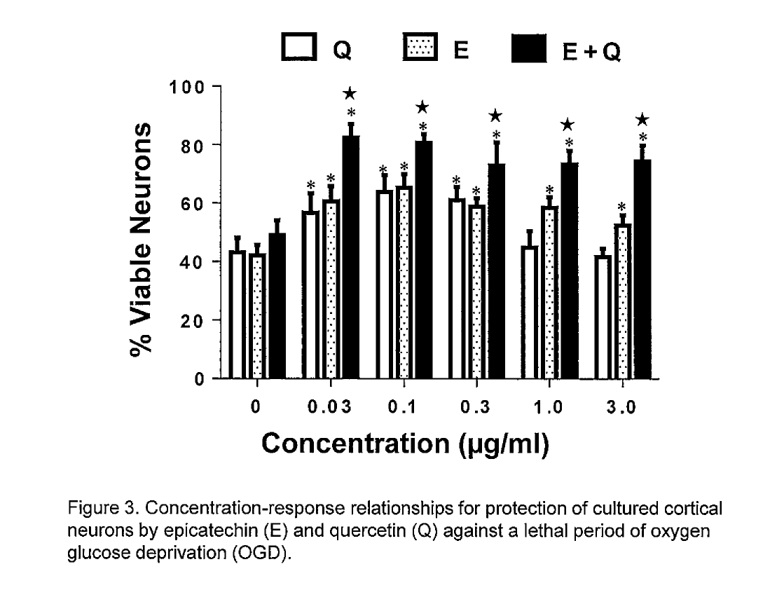

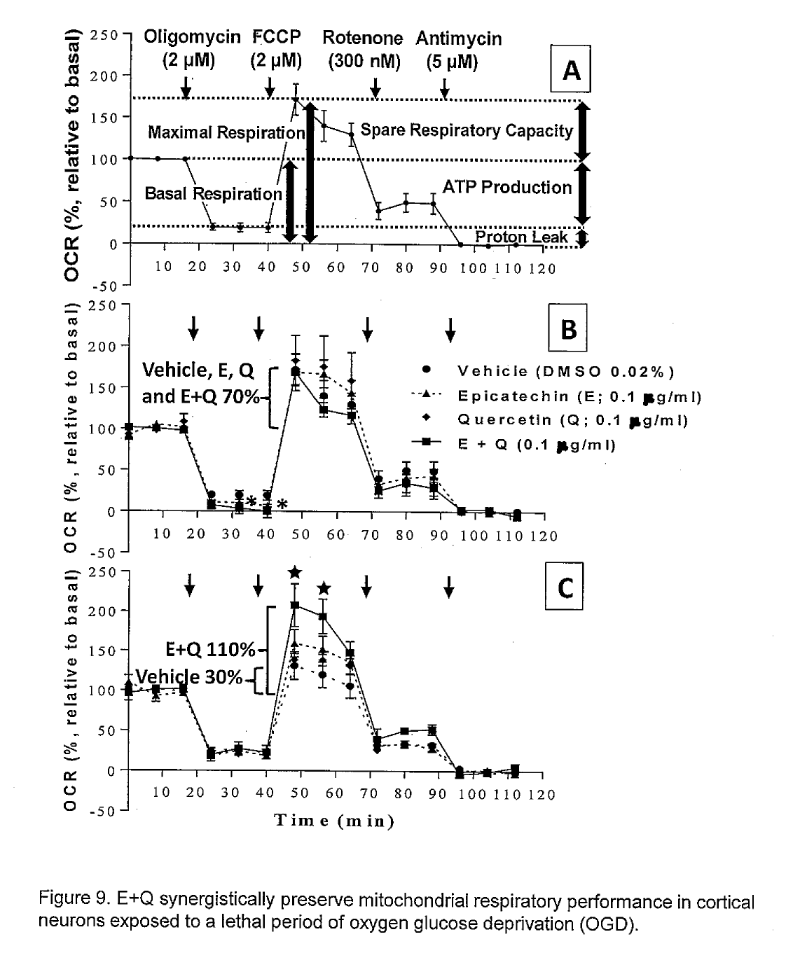

Exposure of cortical neurons to 3 hrs of OGD resulted in a 40% loss of viability 24 hrs later (FIG. 2A). Pretreatment with quercetin (3 μg/ml ˜10 μM) for 24 hrs before OGD (3 hrs) produced a 30% reduction in OGD-induced neuronal cell death (FIG. 2B). By comparison, combining epicatechin (0.5 μg/ml) with quercetin (0.5 μg/ml; total flavonoid concentration of 1 μg/ml) reduced OGD-induced neuronal cell death by over 85% (FIG. 2B). The protective effects of epicatechin plus quercetin against OGD-induced death were not enhanced further by mixing these compounds with other common dietary phenolic acids such as cyanidin and chlorogenic acid (FIG. 2B). Relative to the effects of pretreatment with either epicatechin (1.0 μg/ml ˜3 μM) or quercetin (1.0 μg/ml ˜3 μM) for 24 his before OGD, pre-exposed to epicatechin plus quercetin (1.0 μg/ml) produced supra-additive reductions of 65% in OGD-induced cell death (FIG. 2C). Increasing the concentration of epicatechin plus quercetin (3.0 μg/ml) generated a further decrease of 85% in OGD-induced cell death (FIG. 2C). The protective effects of epicatechin plus quercetin (1.0 μg/ml) were reduced if treatment was delayed until after OGD (FIG. 2D). However, at concentrations of 10 μg/ml or 30 μM, no synergy was observed. At concentrations of 10 μg/ml or above quercetin or epicatechin or epicatechin plus quercetin were increasingly toxic to primary cultures of mouse cortical neurons. Thus, the upper limit for the therapeutic range 3 μg per ml or 10 μM. Detailed concentration-response relationships for neuroprotection by E, Q and E+Q against OGD were performed using the more sensitive method of Fluorescence Activated Cell Sorting (FACS) to count viable neurons (FIG. 3). Relative to control cultures (no OGD), neuronal viability was reduced to approximately 45% by only 90 min of OGD. Moreover, sub-μM concentrations of either E or Q (0.1 or 0.3 μM) were sufficient to increase neuronal survival to 55%. By comparison, equivalent concentrations of E+Q produced much larger increases in viable neurons (˜85%). Isobolograrri analyses of the resultant concentration-cell viability response data for E, Q and E+Q generated a combination index (CI)=0.7 for E+Q (synergism: CI<1.0) [42], confirming that pretreatment with E+Q synergistically protected cortical neuron cultures against OGD. The majority of cell-based studies have used concentrations of 5-10 μM of either E or Q to reduce the death of cultured neurons exposed to excitotoxins [38;108-110], free radical generators [110-114], mitochondrial toxins [115-117] or OGD [38;39;41;57;110;118-120]. Oral administration of these compounds to rodents at doses equivalent to amounts that appear to reduce the risk of neurodegenerative disorders [69;121;122] are neuroprotective in models for stroke, Alzheimer's disease and MS, but only produce brain concentrations in the sub-μM range (0.1-0.4 μM) [123-127]. The relevance of findings from neuronal cell culture studies that have utilised flavonoid concentrations above 1 μM to assess their neuroprotective effects in vivo is therefore questionable [9;121;128-130]. Hence, we have examined the effects of E and Q on cultured neurons at physiologically relevant concentrations (0.1-0.3 μM). E+Q robustly protected cortical neurons against damage after OGD at just 0.1 μM (FIG. 3). Furthermore, neuroprotection by sub-micromolar concentrations of E+Q was closely associated with preserved mitochondrial function (FIG. 9). These findings suggest that at sub-μM concentrations achieved in the brain following oral administration of E+Q, mitochondria play a pivotal role in mediating their neuroprotective effects.

Combining Epicatechin with Quercetin Synergistically Increased Oscillations in Cytosolic Calcium Concentrations in Primary Cultures of Mouse Cortical Neurons

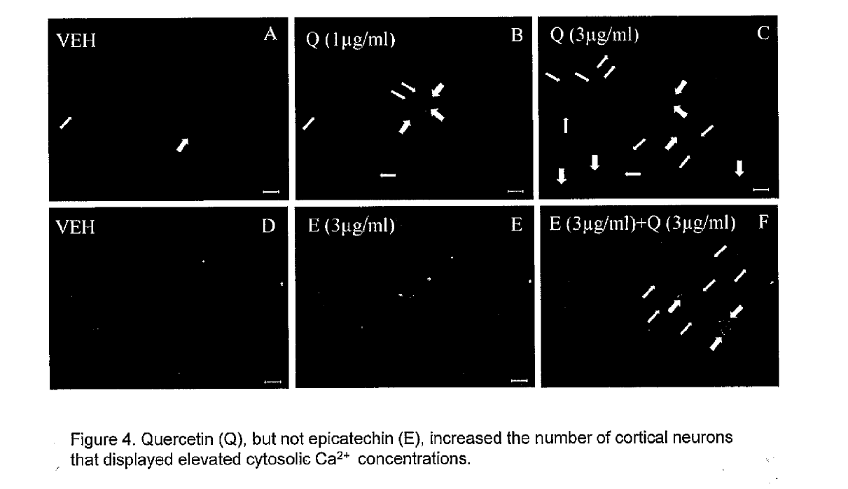

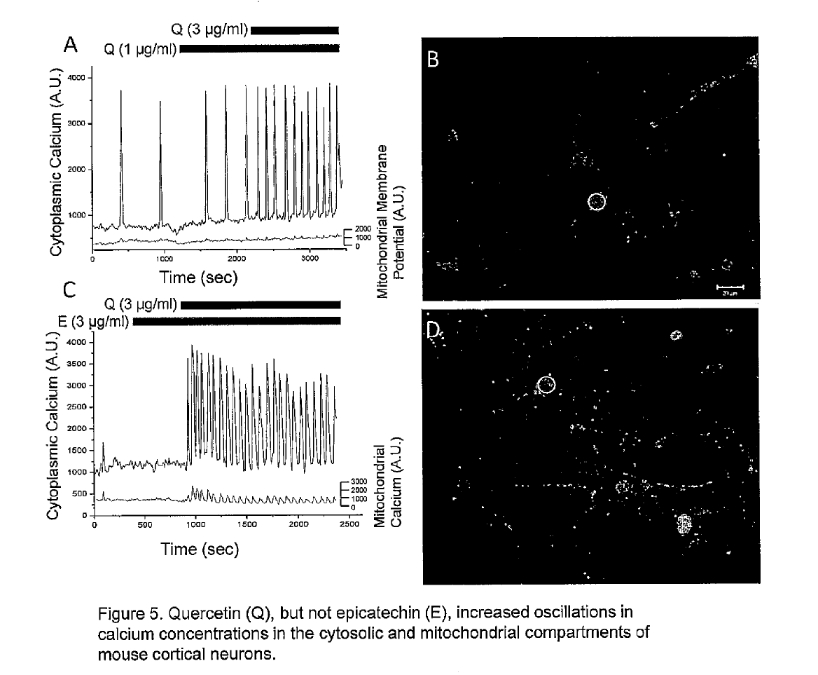

Primary cultures of rodent cortical neurons display spontaneous oscillations in cytosolic calcium concentrations ([Ca²⁺]_{c}) indicative of synaptic activity [131;132]. Treatments that increase synaptic activity render neurons more resistant to ischemic cell death [80;81]. Confocal microscopy revealed that relative to the effects of vehicle (DMSO 0.02%), quercetin (1 and 3 μg/ml ˜3 and 10 μM) increased the number of cortical neurons that displayed [Ca²⁺]_{c} oscillations in a concentration-dependent manner (FIG. 4A-C). Although epicatechin (3 μg/ml ˜10 μM) protected cortical neurons against OGD-induced cell death, this concentration of epicatechin did not increase the number of neurons that displayed [Ca²⁺]_{c} oscillations (FIG. 4D, E). However, the further addition of quercetin (3 μg/ml ˜10 μM) increased the number of neurons that displayed [Ca²⁺]_{c} oscillations (FIG. 4F). Quercetin (1 and 3 μg/ml ˜3-10 μM) also increased the frequency of neuronal [Ca²⁺]_{c} oscillations in a concentration-dependent manner (FIG. 5A). By contrast, epicatechin (3 μg/ml ˜10 μM) did not increase the number of neuronal [Ca²⁺], oscillations (FIG. 5C). However, the further addition of quercetin (3 μg/ml ˜10 μM) produced a supra-additive increase in the number of [Ca²⁺]_{c} oscillations in cortical neurons (FIG. 5C). These results demonstrate that epicatechin and quercetin produced synergistic increases in [Ca²⁺]_{c} oscillations indicative of elevated synaptic activity which is known to initiate signaling events that protect neurons against OGD-induced cell death [80;81].

### Epicatechin and Quercetin Stimulated Mitochondrial Calcium Oscillations and ETC Gene Expression in Cultured Cortical Neurons

The rise in [Ca²⁺]_{c} associated with increased neuronal activity is accompanied by an elevation in mitochondrial calcium uptake [133;134]. Mitochondria calcium entry rapidly stimulates oxidative phosphorylation enabling neurons to meet the dynamic energy demands placed on these excitable cells [135-137]. The mitochondrial calcium uniporter (MCU) is an important mechanism for mitochondrial calcium uptake in neurons [138;139]. Flavonoids including kaempferol and quercetin increase mitochondrial calcium uptake by activating the MCU [83;140]. This MCU-mediated increase in mitochondrial calcium concentrations ([Ca²⁺]ₘ) is thought to promote neuronal activity by enhancing energy production [141;142]. In keeping with a close link between [Ca²⁺]ₘ and neuronal activity, the synergistic increases in [Ca²⁺]ₗ oscillations produced by epicatechin and quercetin (3 μg/ml ˜10 μM) were accompanied by the induction of [Ca²⁺]ₘ oscillations in cortical neurons (FIG. 5C). The induction of [Ca²⁺]ₘ oscillations were accompanied by elevations in mRNA levels for respiratory genes of the ETC encoding members of Complex I (NADH-ubiquinone oxidoreductase chain 2; MT-ND2), Complex II (succinate dehydrogenase, SDHA: nuclear encoded), Complex III (cytochrome b; MT-CytB) and Complex V (ATP synthase F0 subunit 6; ATP6) (FIG. 7). Furthermore, pretreatment with epicatechin and quercetin (1 or 3 μg/ml ˜3 or 10 μM) for 24 hrs before OGD produced dose-dependent increases in the induction of these genes by OGD as well as cytochrome c oxidase subunit II (MT-CO2), a component of Complex IV (FIG. 7). These findings indicate that the profound protection against OGD achieved by combining epicatechin and quercetin (3 μg/ml ˜10 μM) was closely associated with enhanced mitochondrial calcium uptake and ETC gene expression indicative of improved performance of this organelle.

### Epicatechin and Quercetin Promoted Pro-Survival Alterations in Apoptotic Gene Expression in Cultured Cortical Neurons

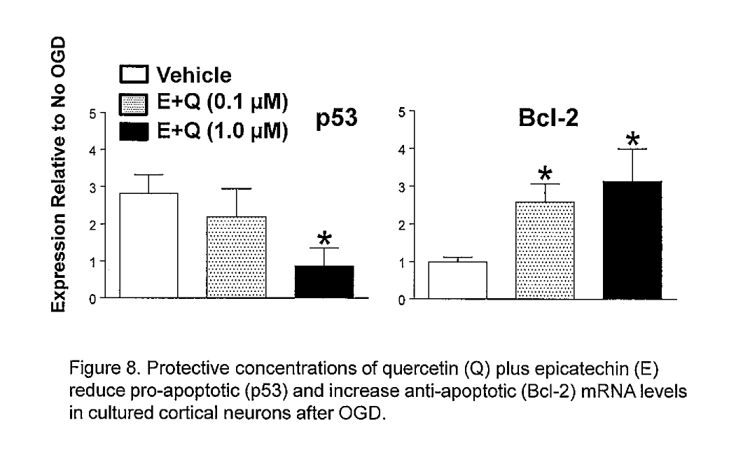

Epicatechin (E) plus quercetin (Q) produced beneficial changes in apoptotic gene expression in cortical neurons after oxygen glucose deprivation (OGD). Consistent with the induction of p53-mediated apoptosis by OGD [45], p53 mRNA levels were elevated 12 hr after OGD relative to control cultures (no OGD) (FIG. 8A). E+Q reversed these increases in a concentration-dependent manner. Neuroprotection by E+Q was also accompanied by increased mRNA levels for the anti-apoptotic gene Bcl-2 (FIG. 8B). Since cAMP-response element binding protein (CREB) promotes neuronal survival by directly activating CRE elements that drive Bcl-2 expression [46-48], this observation indicates that CREB plays an important role in neuroprotection by E+Q.

### Combination Treatment Protects Against Cisplatin-Induced Ototoxicity and Hearing Loss

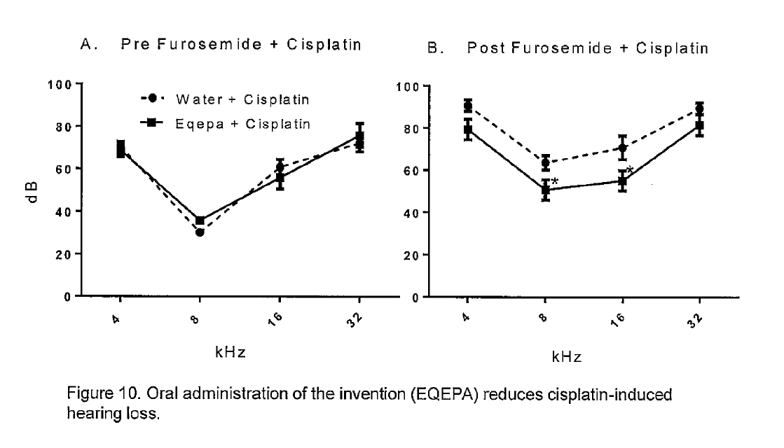

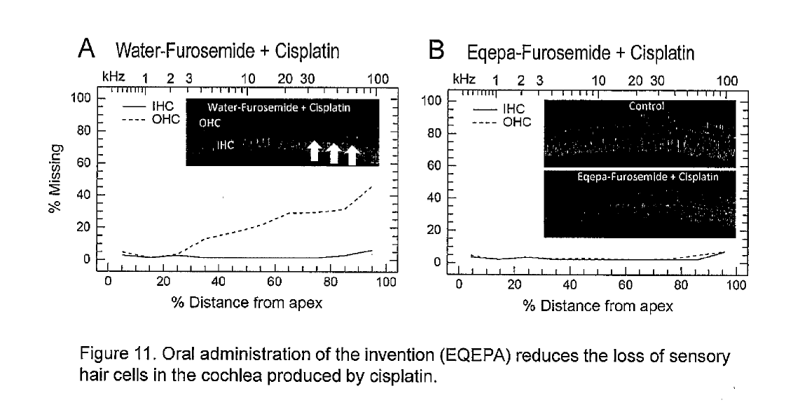

Cisplatin is a widely used chemotherapeutic that produces hearing loss in over 90% of patients treated with this potent antineoplastic agent [143]. Mice given cisplatin also suffer sensory hair cell death in the cochlea (ototoxicity) producing hearing loss [144]. Mortality caused by kidney failure in mice injected with cisplatin is reduced by co-administration of the loop-diuretic furosemide [107]. By contrast, cisplatin-induced hearing loss is potentiated by co-administration of furosemide [107]. Relative to mice treated daily by oral gavage (p.o.) with water (100 μl/mouse), mice that received the composition (EQEPA 100 μl/mouse, p.o.; once daily) beginning 7 days before intraperitoneal (i.p.) injection of furosemide (200 mg/kg) followed by cisplatin (0.2 mg/kg, i.p.) displayed a marked reduction in hearing loss 7 days later (FIG. 10). Cell counts of sensory hair cells in the cochlea revealed that the composition-mediated protection against hearing loss was accompanied by a reduction in sensory hair cell loss in the cochlea (FIG. 11).

### Combination Treatment Attenuated Cisplatin-Induced Hearing Loss in Mice

Prior to the injection of furosemide (200 mg/kg, i.p.) and cisplatin (0.2 mg/kg, i.p.), ABRs measured at 4, 8, 16 and 32 kHz were the same for mice allocated to the two treatment groups: water (100 μl, p.o.; once daily for 14 days) or the composition of the invention (EQEPA 100 μl, p.o.; once daily for 14 days) (FIG. 10A). Administration of water (100 μl, p.o.; once daily for 14 days) or the composition (EQEPA 100 μl, p.o.; once daily) began 7 days before injection of injection of furosemide (200 mg/kg, i.p.) and cisplatin (0.2 mg/kg, i.p.) and ended 7 days later. ABRs recorded 7 days after the injection of furosemide (200 mg/kg, i.p.) and cisplatin (0.2 mg/kg, i.p.) revealed water-treated mice displayed greater hearing loss relative to mice treated with the composition of the invention at 8 and 16 kHz (FIG. 10B).

### Reduced Hypoxic-Ischemic Brain Injury by Oral Administration of the Invention

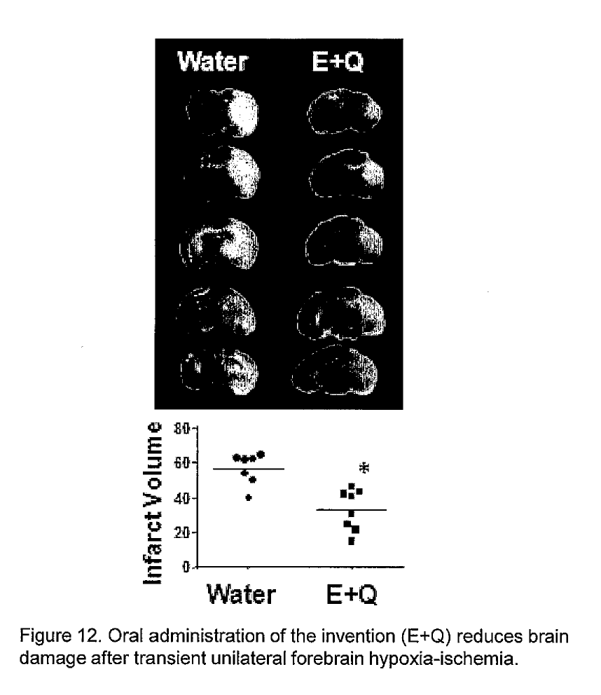

Findings from the OGD model were extended by in vivo testing using a mouse model of hypoxic-ischemic (HI) brain injury. The HI model consists of unilateral common carotid artery occlusion followed by placement of the animal in a low oxygen (8%) environment to induce hypoxic stress [145]. The combination of unilateral carotid occlusion and hypoxia diminishes cerebral blood flow to levels seen with focal ischemia models causing infarction [146]. Brain injury occurs almost exclusively in the ipsilateral hemisphere that primarily involves the hippocampus and striatum, with some neuronal loss in the surrounding cortex [147-149]. Neuronal loss in the striatum and cortex results in motor deficits that are ameliorated by a variety of neuroprotective treatments [148;150-152], including flavonoids [153]. Administration of either E [57] or Q [58;59;154;155] reduces ischemic brain injury, however, the benefits of combining these compounds have not been reported. Oral administration of the invention once daily for 5 days before HI markedly reduced brain damage (FIG. 12). The degree of neuroprotection produced by combining E and Q (57% reduction in infarct volume) is much greater than that reported for comparable doses of E or Q alone (25-30%) [41;57-59]. These findings suggest that combined administration of E and Q synergistically increases resistance to ischemic brain damage.

### Methods and Materials

### Animal Care

All experiments involving the use of animals were approved by the Dalhousie University Committee on Laboratory Animals and done in accordance with guidelines for the Canadian Council on Animal Care. The animal holding rooms were on a 12-hour dark/light cycle with water and food provided ad libitum.

### Preparation of Mouse Primary Cortical Neuron Cultures

Embryonic day 16 timed pregnant CD1 out-bred mice were obtained from Charles River Laboratories (Charles River; QC, Canada). Primary cortical neuron cultures were prepared from cerebral cortices of wild type (WT) CD1 mouse embryos as described previously [156], with the following modifications. Pregnant CD1 females were heavily anaesthetized with isoflurane vapor (Benson Medical Industries, Inc., Markham, ON) before being euthanized by decapitation. The embryonic day 16 (E16) fetuses were immediately removed from the sacrificed pregnant females by cesarean section and placed in ice-cold Hank's Balanced Salt Solution (HBSS) (GIBCO; Invitrogen, Amarillo, Calif.). The meninges were removed from the brains and cortices were isolated under a dissecting microscope. The cortices from each embryo were placed in individual wells of a 24-well plate (Corning; Lowell, Mass.), containing 1 ml of ice-cold PBS (GIBCO; Invitrogen, Amarillo, Calif.) with 1 mM Mg²⁺, 13 mM glucose and 0.3% w/v bovine serum albumin (BSA) (Invitrogen, Amarillo, Calif.). Under sterile conditions, the tissue was briefly minced, transferred to 15 ml sterile conical tubes (Corning; Lowell, Mass.) and centrifuged at 350×g for 3 min at room temperature. The dissecting solution was discarded and the cortical neurons were then dissociated trituration with fire-polished glass pipettes followed by incubating in 1 ml of 0.1% trypsin solution (0.1% w/v trypsin (Invitrogen, Amarillo, Calif.) in PBS with 1 mM Mg²⁺ and 13 mM glucose) at 37° C. for 15 min. The trypsinization was inhibited by the addition of 0.5 ml of trypsin inhibitor solution that also contained DNase I (0.06% w/v trypsin inhibitor (Invitrogen; Amarillo, Calif.) and 0.01% DNase I (Invitrogen; Amarillo, Calif.) in PBS with 1 mM Mg²⁺, 13 mM glucose and 0.3% w/v BSA). The tubes were mixed briefly and the cells were centrifuged at 350×g for 3 min at room temperature. The trypsin and inhibitor solutions were discarded and each cell pellet was suspended in 1 ml of cortical neuron plating medium (Neurobasal medium (Invitrogen, Amarillo, Calif.) with 10% fetal bovine serum (GIBCO; Invitrogen; Amarillo, Calif.), 2% B27 supplement, 1 mM L-glutamine, and 1% Gentamycin (Invitrogen; Amarillo, Calif.), triturated 10 times and counted using trypan blue exclusion and a hemocytometer. Cortical neurons were plated in 96-well plates (Corning; Lowell, Mass.) that were pre-coated with poly-D-lysine (PDL; Sigma-Aldrich; Oakville, ON) according to the procedure described by the manufacturer. Briefly, plates were coated immediately before use with 100 μg/ml PDL for 5-10 min (50 μl/well), washed three times with tissue-culture grade water and left to dry for 2 h before cells were introduced. Cortical neurons were plated at a concentration of 1×10⁶ cells/ml (100 μ1/well) and medium was completely changed the day after plating to serum-free cortical neuron medium (Neurobasal medium with 2% B27 supplement, 5 mM HEPES, 0.5 mM L-glutamine, and 20 μg/ml Gentamycin), which was replaced every 3 days in culture. Cultures were maintained in a humidified, 37° C. incubator with 5% CO₂. Experiments were performed on the eighth day in vitro (DIV10).

### Confocal Imaging of Calcium Transients

For measurements of mitochondrial membrane potential (ΔΨₘ), cells were loaded with 25 nM tetrannethylrhodamine methyl ester (TMRM) for 30 min at room temperature and the dye was present during the experiment. TMRM is used in the redistribution mode to assess ΔΨₘ with a reduction in TMRM fluorescence indicating mitochondrial depolarization. Neurons were loaded for 30 min at room temperature with X-rhod-1 AM (0.5 μM) and Fluo-3 AM (0.5 μM) (Molecular Probes, Invitrogen) prior to imaging in HEPES-buffered salt solution (HBSS) composed of (mM): 156 NaCl, 3 KCl, 2 MgSO₄, 1.25 KH₂PO₄, 2 CaCl₂, 10 glucose and 10 HEPES, pH adjusted to 7.35 with NaOH. Confocal images were obtained using a 510 CLSM (Zeiss, Thornwood, N.Y.) equipped with a META detection system and a X40 oil-immersion objective. The 488 nm Argon laser line was used to excite fluo-3 fluorescence, which was measured using a bandpass filter from 505 to 550 nm. Illumination intensity was kept to a minimum (at 0.1-0.2% of laser output) to avoid phototoxicity and the pinhole was set to give an optical slice of ˜2 μm. TMRM and X-rhod-1 were excited using the 543 nm laser line with fluorescence measured using a 560 nm longpass filter. All the imaging data are representative of at least 3 experiments.

### Oxygen Glucose Deprivation

Cortical neuron cultures (DIV10) were exposed to 1 μg/ml or 3 μg/ml of epicatechin, quercetin, epicatechin plus quercetin or the corresponding DMSO control (0.02% DMSO) in serum-free cortical neuron medium for 24 hr proceeding as well as during the 24 hr period after OGD on DIVIO. Glucose-free medium (glucose-free Dulbecco's Modified Eagle Medium (GBSS, Invitrogen; Amarillo, Calif.) containing either epicatechin or quercetin or epicatechin plus quercetin at concentration of 1 μg/ml or 3 μg/ml or the corresponding DMSO control (0.02% DMSO) was placed in a 96-well plate and equilibrated to 0% oxygen in a modular chamber incubator (Billups-Rothenberg; Del Mar, Calif.). The chamber was flushed for 4 min at 20 l/min with an anoxic gas mixture (5% CO₂ and Balanced N₂) (PraxAIR; Dartmouth, NS) using a step-down pressure system and placed in a humidified, 37° C. incubator for 12 h. Cortical neuron medium was replaced with OGD-medium (anoxic and glucose-free) and the cultures were placed in the modular chamber incubator. The chamber was flushed again with anoxic gas and placed inside a humidified, 37° C. incubator for 3 hrs. OGD was terminated by removal of the anoxic GBSS and replaced with B27 supplement Neurobasal media that did not contain antioxidants. Cell viability was assessed 24 hours after the completion of OGD.

### Cell Viability

The culture media was exchanged for MEM media without phenol red containing 0.5 mg/ml MTT (3-[4,5-dimethylthiazol-2-yl]-2,5-diphenyltetrazolium bromide). The cells were then placed back into the incubator for approximately 3 hours. Following the 3 hour incubation the media was removed and the insoluble formazan crystals were dissolved by the addition of 200 μl of 90% isopropanol and 10% triton X-100 solution acidified with a few drops of HCl. The absorbance was then measured at 562 nm with a plate reader. FAGS (fluorescence-activated cell sorting) was employed to count the number of viable neurons. Cortical neurons were pretreated with vehicle (DMSO 0.02%; 0 μg/ml) or quercetin (Q) or epicatechin (E) or E+Q at a concentration of 0.03, 0.1, 0.03, 1 or 3 μg/ml for 24 hrs before 3 hours of OGD. Twenty-four hours after oxygen-glucose deprivation, cortical neuron cultures were incubated with Annexin V and 7-Aminoactinomycin D (7AAD). Cytometric analyses was then performed to identify viable (no staining), apoptotic [Annexin (+)], necrotic [7AAD (+)] and late apoptotic (double labelled) cells using a FACSArialll instrument (Becton Dickinson Canada). A scatter plot was employed to exclude by size all cellular debris from the target cell population (P1 gate). A quadrant gate was then applied to this P1 population in order to identify four types of cells: healthy or viable (no stain), apoptotic [Annexin V (+) identified with the PE detector (585/15; 556LP)], necrotic [7AAD (+) identified with the APC detector (660/20)] and late apoptotic (double stained) cells.

### RNA Extraction and Quality Control

Total RNA was extracted from primary cultures of mouse cortical neurons using the QIAGEN RNeasy Plus Mini Kit according to the manufactures instructions. Genomic DNA was removed by the use of a genomic DNA eliminator column provided (QIAGEN) followed by RNAse-free DNAse treatment (QIAGEN). To measure the quality and overall purity of isolated total RNA, an Experion bioanalyzer equipped with a RNA StdSens Analysis Kit was used (Bio-Rad, Hercules, Calif., USA). An Epoch (Biotek, Vt., USA) microplate reader was used to measure the final concentration of total RNA.

### Quantitative RT-PCR

Total RNA (750 ng) was reverse transcribed using the iScript Reverse Transcription kit from BioRad according to the manufactures instructions. Gene analysis was performed using the Sso Fast EvaGreen Supermix kit (BioRad) according to manufacturer's instructions on a BioRad CFX96 Real-Time System. Enzyme activation was carried out at 95° C. for 30 seconds. Denaturation and annealing were both performed for 5 seconds with 95 and 55-60° C. respectively. All genes were run for 40 cycles. The presence of only one product was confirmed after each run by incorporating a melting curve over the range of 65-95° C. Samples were completed in triplicate. Gene expression analysis was completed in accordance with MIQE guidelines. Genes were normalized against β-2-microglobulin and glyceraldehyde 3-phosphate dehydrogenase. Quantitative reverse-transcription polymerase chain reaction (qRT-PCR) was performed using the following primers obtained from Invitrogen: β-2-microglobulin F: TTCTGGTGCTTGTCTCACTGA (SEQ ID No:1) R: CAGTATGTTCGGCTTCCCATTC (SEQ ID No:2); Glyceraldehyde 3-phosphate dehydrogenase F: AGGTCGGTGTGAACGGATTTG (SEQ ID No:3) R: GGGGTCGTTGATGGCAACA (SEQ ID No:4); Mitochondrial ATP synthase subunit 6 F: AATTACAGGCTTCCGACACAAAC (SEQ ID No:5) R: TGGAATTAGTGAAATTGGAGTTCCT (SEQ ID No:6); NADH-ubiquinone oxido-reductase chain 2 F: GGGCATGAGGAGGACTTAACCAAAC (SEQ ID No:7) R: TGAGGTTGAGTAGAGTGAGGGATGG (SEQ ID No:8); Succinate dehydrogenase subunit A F: TGTTCAGTTCCACCCCACA (SEQ ID No:9) R: TCTCCACGACACC CTTCTGT (SEQ ID No:10); Mitochondrial cytochrome oxidase subunit 2 F: CAGTATATTCGTAGCTTCAG (SEQ ID No:11) R: CCTCTAATCATCTCGCTAA (SEQ ID No:12); Mitochondrial cytochrome b F: CCACTTCATCTTACCATTTATTATCGC (SEQ ID No:13) R: TTTTATCTGCATCTGAGTTTAATCCTGT (SEQ ID No: 14); PGC1-α F: CAATGAATGCAGCGGTCTTA (SEQ ID No:15) R: GTGTGAGGAGGGTCATCGTT (SEQ ID No:16). P53 F: TACTCTCCTCCCCTCAATAA (SEQ ID No:17) R: CTTGTAGTGGATGGTGGTAT (SEQ ID No:18); BCL2 F: ATGCCTTTGTGGAACTATATGGC (SEQ ID No:19) R: GGTATGCACCCAGAGTGATGC (SEQ ID No:20). Results are expressed as fold changes relative to a matched calibrator sample extracted from control cortical neurons not exposed to OGD. Relative changes from the calibrator were calculated using the 2^{-ΔΔCt} method [157].

### Measurement of Mitochondrial Respiration in Primary Cultures of Mouse Cortical Neurons

The effects of E, Q and E+Q on key parameters of mitochondrial respiration in primary cultures of mouse cortical neurons were measured using a Seahorse Bioscience XF24 Extracellular Flux Analyzer XF24 instrument. Oxygen consumption rate (OCR) in fmol/cell were normalized by counting viable cortical neurons at the end of each experiment. A seeding density of 80,000 neurons/well was sufficient to obtain a measurable basal OCR (4-6 fmol/min/cell) permitting both OCR induction and inhibition to be assessed. Mitochondrial function in cortical neurons was measured by the sequential injection of oligomycin (2 μM), FCCP (2 μM), rotenone (300 nM) and antimycin (5 μM) to control and OGD-exposed cultures treated with various concentrations of E, Q or E+Q (0.1 μM).

### Measurement of Auditory Brainstem Responses

Mice were anesthetized by intraperitoneal injection of a mixture of ketamine (80 mg/kg) plus xylazine (16 mg/kg). Body temperature was maintained at 37° C. with a heating pad. TuckerDavis hardware and BioSig software [Tucker-Davis Technology (TDT) system III, Alachua, Fla., USA] were used for the signal generation and acquisition of the auditory brainstem responses to tone bursts of 4, 8, 16 and 32 kHz. Each tone burst (10 ms; rise/fall of 1 ms) was delivered through a broadband electrostatic speaker (ESI from TDT) placed 10 cm in front of the animal's head in a sound-proof booth. At each frequency, the signal was presented from 90 dB SPL (sound pressure level) down to 10 dB SPL in 5 dB steps. Auditory brainstem responses (ABRs) were recorded with three electrodes placed subdermally (non-inverted at vertex), reference and grounding electrodes behind the two ears). The electrical responses were collected using TDT hardware (RA16PA and BA) and software (BioSigRP), amplified by 20, filtered between 100 and 3 KHz and averaged 1000 times. The stimuli started at 90 dB and were presented in downward increments of 5 dB. The responses were band-pass filtered between 100-3000 Hz, amplified and averaged over 1000 times with a repetition rate of 21.1 s⁻¹. The threshold was defined as the point at which a repeatable third wave was observed. If no waveform was identified at the highest presentation level (90 dB SPL) for a particular frequency, the threshold was recorded as 100 dB SPL.

### Drug Preparation and Administration

Cisplatin (cis-diamminedichloridoplatinum(II); Sigma) and furosemide [4-chloro-2-(furan-2-ylmethylamino)-5-sulfamoylbenzoic acid] were dissolved in 0.9% saline. Epicatechin (E; 3 mg) and Quercetin (QU; 3 mg) were dissolved in 1.1 g of EPA ethyl ester (96% pure; specific gravity of EPA ethyl ester is 1.11 g/ml) to generate 1 ml of the composition of the invention. Water (100 μl) and the composition of the invention (100 μl) were administered by oral gavage (p.o.). Furosemide (200 mg/kg) was injected by the intraperitoneal (i.p.) route one hour before administration of cisplatin (0.2 mg/kg, i.p.).

### Treatment Groups

On day 0, baseline ABR recordings were performed on 14 adult male C57BI/6 mice. Mice were then randomly assigned to one of two treatment groups composed of 7 animals each. On day 1, one group of mice received water (8 ml/kg, p.o.) while the other group was treated with the composition of the invention (100 μl, pm.). Oral administration of water (100 μl, p.o.; once daily) or the composition of the invention (100 μl, p.o.; once daily) continued until day 14. On day 7, all animals received an injection of furosemide (200 mg/kg, i.p.) followed 1 hr later by cisplatin (0.2 mg/kg, i.p). On day 14, ABR measurements were performed a second time on all mice.

### Cytocochleogram

The cytocochleogram was determined by the spatial percentage count of missing hair cells along the cochlear duct. The mice were deeply anesthetized with an over-dose of ketamine, and the cochleas rapidly harvested after the final ABR test. Surrounding soft tissues were removed, and the round window and oval window were both opened. A small hole was made with a needle at the apex of the cochlea for perfusion and staining. The staining solution for succinate dehydrogenase (SDH) histochemistry was freshly prepared by mixing 0.2M sodium succinate (2.5 ml), phosphate buffered saline (2.5 ml) and nitro-tetranitro blue tetrazolium (nitro-BT, 5 ml). The cochlea was gently perfused through the hole at the cochlear apex and the opened round and oval windows. Following this, the cochlea was immersed in the SDH solution for 45 min at 37° C., and then fixed with 10% formalin for 4 h. After fixation, the cochlea was decalcified with 5% EDTA solution for 72 h. The organ of Corti was dissected and surface preparations were made on slides. Cytocochleogranns were established using normative data for C57BL/6J mice using custom-written software.

### Hypoxic-Ischemic Brain Damage

Mice were anaesthetized using isoflurane (Baxter Corporation; Mississauga, ON, Canada) in an induction chamber (3% vaporized with medical oxygen at a flow rate of 3 L/min). The ventral portion of the neck was shaved and then sterilized with Soluprep (SoluMed Inc.; Laval, QC, Canada) and Betadine (Purdue Frederick Inc.; Pickering, ON). Anesthesia was maintained with 2% isoflurane vaporized with oxygen at a flow rate of 1.5 L/min. A small ventral incision was made on the neck of the mouse with a pair of scissors to expose the sternohyoid and sternomastoid muscles. The left carotid artery was located beneath the intersection point of the sternohyoid and the sternomastoid muscles. The left carotid artery was carefully separated from the vagus nerve and permanently occluded using a high-temp electrocautery pen (Boyle Instruments; St. Petersburg, Fla.). Following a 2-3 h recovery period the mice were placed in a hypoxia-chamber, consisting of a glass cylinder vented with 8% oxygen balanced with nitrogen flowing at a rate of 6 L/min. The chamber was placed in a water bath at 36.5° C. to maintain normal body temperature. After 40 min of exposure to the low oxygen environment (8% oxygen balanced with nitrogen) mice were removed from the chamber and returned to their home cage. The mice were allowed to survive for 24 hr following HI to permit the brain infarct in the ipsilateral hemisphere to develop. Infarct volume was measured 24 hr later by staining of serial brain sections with the vital dye triphenyl tetrazolium chloride (TTC). Volumetric measures of each hemisphere were carried out to determine infarct size. The area of the infracted hemisphere was measured using the tracing function in Scion image on serial sections 1 mm thick between Bregma 1.18 mm and −2.80 mm and a volume between sections was approximated by multiplying the area by 1 mm (the distance between consecutive sections).

### Treatment Groups

Two groups of adult C57BI/6 mice, composed of 7-8 animals each, were dosed orally with either the composition of the invention (100 μl, p.o.) or water (100 μl) once daily. Five days later, all mice were subjected to unilateral forebrain hypoxia-isvchemia (40 min). Infarct volume was measured 24 hr later by staining of serial brain sections with the vital dye triphenyl tetrazolium chloride (TIC) (14 adult male C57BI/6 mice.

The scope of the claims should not be limited by the preferred embodiments set forth in the examples, but should be given the broadest interpretation consistent with the description as a whole.

### REFERENCE LIST

[1] S. B. Vafai, V. K. Mootha, Mitochondrial disorders as windows into an ancient organelle, Nature 491 (2012) 374-383.

[2] P. Racay, Z. Tatarkova, M. Chomova, J. Hatok, P. Kaplan, D. Dobrota, Mitochondrial calcium transport and mitochondrial dysfunction after global brain ischemia in rat hippocampus, Neurochem. Res. 34 (2009) 1469-1478.

[3] Z. V. Niatsetskaya, S. A. Sosunov, D. Matsiukevich, I. V. Utkina-Sosunova, V. I. Ratner, A. A. Starkov, V. S. Ten, The oxygen free radicals originating from mitochondrial complex I contribute to oxidative brain injury following hypoxia-ischemia in neonatal mice, J. Neurosci. 32 (2012) 3235-3244.

[4] M. Chomova, Z. Tatarkova, D. Dobrota, P. Racay, Ischemia-induced inhibition of mitochondrial complex I in rat brain: effect of permeabilization method and electron acceptor, Neurochem. Res. 37 (2012) 965-976.

[5] S. Drose, U. Brandt, Molecular mechanisms of superoxide production by the mitochondrial respiratory chain, Adv. Exp. Med. Biol. 748 (2012) 145-169.

[6] R. K. Chaturvedi, B. M. Flint, Mitochondrial diseases of the brain, Free Radio. Biol. Med. 63 (2013) 1-29.

[7] M. T. Lin, M. F. Beal, Mitochondrial dysfunction and oxidative stress in neurodegenerative diseases, Nature 443 (2006) 787-795.

[8] A. P. Halestrap, Calcium, mitochondria and reperfusion injury: a pore way to die, Biochem. Soc. Trans. 34 (2006) 232-237.

[9] Q. R. Jones, J. Warford, H. P. Rupasinghe, G. S. Robertson, Target-based selection of flavonoids for neurodegenerative disorders, Trends Pharmacol. Sci. 33 (2012) 602-610.

[10] R. Dutta, J. McDonough, X. Yin, J. Peterson, A. Chang, T. Torres, T. Gudz, W. B. Macklin, D. A. Lewis, R. J. Fox, R. Rudick, K. Mirnics, B. D. Trapp, Mitochondrial dysfunction as a cause of axonal degeneration in multiple sclerosis patients, Ann. Neurol. 59 (2006) 478-489.

[11] N. Exner, A. K. Lutz, C. Haass, K. F. Winklhofer, Mitochondrial dysfunction in Parkinson's disease: molecular mechanisms and pathophysiological consequences, EMBO J. 31(2012) 3038-3062.

[12] R. K. Chaturvedi, M. F. Beal, Mitochondrial approaches for neuroprotection, Ann. N. Y. Acad. Sci. 1147 (2008) 395-412.

[13] B. Munos, Lessons from 60 years of pharmaceutical innovation, Nat. Rev. Drug Discov. 8 (2009) 959-968.

[14] A. L. Frazier, C. Weldon, J. Amatruda, Fetal and neonatal germ cell tumors, Semin. Fetal Neonatal Med. 17 (2012) 222-230.

[15] Y. Jin, X. Y. Cai, Y. X. Shi, X. Y. Xia, Y. C. Cai, Y. Cao, W. D. Zhang, W. H. Hu, W. Q. Jiang, Comparison of five cisplatin-based regimens frequently used as the first-line protocols in metastatic nasopharyngeal carcinoma, J. Cancer Res. Clin. Oncol. 138 (2012) 1717-1725.

[16] M. B.Lustberg, M. J. Edelman, Optimal duration of chemotherapy in advanced non-small cell lung cancer, Curr. Treat. Options. Oncol. 8 (2007) 38-46.

[17] G. A. Omura, Progress in gynecologic cancer research: the Gynecologic Oncology Group experience, Semin. Oncol. 35 (2008) 507-521.

[18] M. E. Randall, V. L. Filiaci, H. Muss, N. M. Spirtos, R. S. Mannel, J. Fowler, J. T. Thigpen, J. A. Benda, Randomized phase III trial of whole-abdominal irradiation versus doxorubicin and cisplatin chemotherapy in advanced endometrial carcinoma: a Gynecologic Oncology Group Study, J. Clin. Oncol. 24 (2006) 36-44.

[19] B. H. Zoltick, Shedding light on testicular cancer, Nurse Pract. 36 (2011) 32-39.

[20] G. Ciarimboli, D. Deuster, A. Knief, M. Sperling, M. Holtkamp, B. Edemir, H. Pavenstadt, C. Lanvers-Kaminsky, A. am Zehnhoff-Dinnesen, A. H. Schinkel, H. Koepsell, H. Jurgens, E. Schlatter, Organic cation transporter 2 mediates cisplatin-induced oto- and nephrotoxicity and is a target for protective interventions, Am. J. Pathol. 176 (2010) 1169-1180.

[21] L. P. Rybak, C. A. Whitworth, D. Mukherjea, V. Ramkumar, Mechanisms of cisplatin-induced ototoxicity and prevention, Hear. Res. 226 (2007) 157-167.

[22] N. Fischel-Ghodsian, R. D. Kopke, X. Ge, Mitochondrial dysfunction in hearing loss, Mitochondrion. 4 (2004) 675-694.

[23] H. J. Kim, J. H. Lee, S. J. Kim, G. S. Oh, H. D. Moon, K. B. Kwon, C. Park, B. H. Park, H. K. Lee, S. Y. Chung, R. Park, H. S. So, Roles of NADPH oxidases in cisplatin-induced reactive oxygen species generation and ototoxicity, J. Neurosci. 30 (2010) 3933-3946.

[24] M. J. McKeage, Comparative adverse effect profiles of platinum drugs, Drug Saf 13 (1995) 228-244.

[25] C. F. Pollera, P. Marolla, M. Nardi, F. Ameglio, L. Cozzo, F. Bevere, Very high-dose cisplatin-induced ototoxicity: a preliminary report on early and long-term effects, Cancer Chemother. Pharmacol. 21(1988) 61-64.

[26] A. Yancey, M. S. Harris, A. Egbelakin, J. Gilbert, D. B. Pisoni, J. Renbarger, Risk factors for cisplatin-associated ototoxicity in pediatric oncology patients, Pediatr. Blood Cancer (2012).

[27] C. J. Ross, H. Katzov-Eckert, M. P. Dube, B. Brooks, S. R. Rassekh, A. Barhdadi, Y. Feroz-Zada, H. Visscher, A. M. Brown, M. J. Rieder, P. C. Rogers, M. S. Phillips, B. C. Carleton, M. R. Hayden, Genetic variants in TPMT and COMT are associated with hearing loss in children receiving cisplatin chemotherapy, Nat. Genet. 41(2009) 1345-1349.

[28] X. Huang, C. A. Whitworth, L. P. Rybak, Ginkgo biloba extract (EGb 761) protects against cisplatin-induced ototoxicity in rats, Otol. Neurotol. 28 (2007) 828-833.

[29] S. J. Choi, S. W. Kim, J. B. Lee, H. J. Lim, Y. J. Kim, C. Tian, H. S. So, R. Park, Y. H. Choung, Gingko biloba extracts protect auditory hair cells from cisplatin-induced ototoxicity by inhibiting perturbation of gap junctional intercellular communication, Neuroscience 244 (2013) 49-61.

[30] S. L. Zeiger, J. N. Stankowski, B. McLaughlin, Assessing neuronal bioenergetic status, Methods Mal. Biol. 758 (2011) 215-235.

[31] A. G. Herrmann, R. F. Deighton, B. T. Le, M. C. McCulloch, J. L. Searcy, L. E. Kerr, J. McCulloch, Adaptive changes in the neuronal proteome: mitochondrial energy production, endoplasmic reticulum stress, and ribosomal dysfunction in the cellular response to metabolic stress, J. Cereb. Blood Flow Metab 33 (2013) 673-683.

[32] Y. Zhu, P. Hoell, B. Ahlemeyer, U. Sure, H. Bertalanffy, J. KriegIstein, Implication of PTEN in production of reactive oxygen species and neuronal death in in vitro models of stroke and Parkinson's disease, Neurochem. Int. 50 (2007) 507-516.

[33] A. Kichev, C. I. Rousset, A. A. Baburamani, S. W. Levison, T. L. Wood, P. Gressens, C. Thornton, H. Hagberg, TNF-related apoptosis-inducing ligand (TRAIL) signaling and cell death in the immature central nervous system after hypoxia-ischernia and inflammation, J. Biol. Chem. (2014).

[34] Y. Zhang, P. Lipton, Cytosolic Ca2+ changes during in vitro ischemia in rat hippocampal slices: major roles for glutamate and Na+-dependent Ca2+ release from mitochondria, J. Neurosci. 19 (1999) 3307-3315.

[35] D. G. Nicholls, Mitochondrial dysfunction and glutamate excitotoxicity studied in primary neuronal cultures, Curr. Mol. Med. 4 (2004) 149-177.

[36] M. Papadakis, G. Hadley, M. Xilouri, L. C. Hoyte, S. Nagel, M. M. McMenamin, G. Tsaknakis, S. M. Watt, C. W. Drakesmith, R. Chen, M. J. Wood, Z. Zhao, B. Kessler, K. Vekrellis, A. M. Buchan, Tsc1 (hamartin) confers neuroprotection against ischemia by inducing autophagy, Nat. Med. 19 (2013) 351-357.

[37] J. D. Sinor, S. Du, S. Venneti, R. C. Blitzblau, D. N. Leszkiewicz, P. A. Rosenberg, E. Aizenman, NMDA and glutamate evoke excitotoxicity at distinct cellular locations in rat cortical neurons in vitro, J. Neurosci. 20 (2000) 8831-8837.

[38] H. J. Ha, Y. S. Kwon, S. M. Park, T. Shin, J. H. Park, H. C. Kim, M. S. Kwon, Quercetin attenuates oxygen-glucose deprivation- and excitotoxin-induced neurotoxicity in primary cortical cell cultures, Biol. Pharm. Bull. 26 (2003) 544-546.

[39] R. L. Liu, Q. J. Xiong, Q. Shu, W. N. Wu, J. Cheng, H. Fu, F. Wang, J. G. Chen, Z. L. Hu, Hyperoside protects cortical neurons from oxygen-glucose deprivation-reperfusion induced injury via nitric oxide signal pathway, Brain Res. 1469 (2012) 164-173.

[40] K. S. Panickar, M. M. Polansky, R. A. Anderson, Green tea polyphenols attenuate glial swelling and mitochondrial dysfunction following oxygen-glucose deprivation in cultures, Nutr. Neurosci. 12 (2009) 105-113.

[41] C. C. Leonardo, M. Agrawal, N. Singh, J. R. Moore, S. Biswal, S. Dore, Oral administration of the flavanol (−)-epicatechin bolsters endogenous protection against focal ischemia through the Nrf2 cytoprotective pathway, Eur. J. Neurosci. 38 (2013) 3659-3668.

[42] L. Zhao, M. G. Wientjes, J. L. Au, Evaluation of combination chemotherapy: integration of nonlinear regression, curve shift, isobologram, and combination index analyses, Clin. Cancer Res. 10 (2004) 7994-8004.

[43] K. M. Holmstrom, L. Baird, Y. Zhang, I. Hargreaves, A. Chalasani, J. M. Land, L. Stanyer, M. Yamamoto, A. T. Dinkova-Kostova, A. Y. Abramov, Nrf2 impacts cellular bioenergetics by controlling substrate availability for mitochondrial respiration, Biol. Open. 2 (2013) 761-770.

[44] G. Hajnoczky, L. D. Robb-Gaspers, M. B. Seitz, A. P. Thomas, Decoding of cytosolic calcium oscillations in the mitochondria, Cell 82 (1995) 415-424.

[45] C. Culnnsee, J. Siewe, V. Junker, M. Retiounskaia, S. Schwarz, S. Camandola, S. El-Metainy, H. Behnke, M. P. Mattson, J. KriegIstein, Reciprocal inhibition of p53 and nuclear factor-kappaB transcriptional activities determines cell survival or death in neurons, J. Neurosci. 23 (2003) 8586-8595.

[46] T. Mabuchi, K. Kitagawa, K. Kuwabara, K. Takasawa, T. Ohtsuki, Z. Xia, D. Storm, T. Yanagihara, M. Hori, M. Matsumoto, Phosphorylation of cAMP response element-binding protein in hippocampal neurons as a protective response after exposure to glutamate in vitro and ischemia in vivo, J. Neurosci. 21(2001) 9204-9213.

[47] W. Y. Lin, Y. C. Chang, H. T. Lee, C. C. Huang, CREB activation in the rapid, intermediate, and delayed ischemic preconditioning against hypoxic-ischemia in neonatal rat, J. Neurochem. 108 (2009) 847-859.

[48] K. Kitagawa, CREB and cAMP response element-mediated gene expression in the ischemic brain, FEBS J. 274 (2007) 3210-3217.

[49] B. P. Dranka, B. G. Hill, V. M. Darley-Usnnar, Mitochondrial reserve capacity in endothelial cells: The impact of nitric oxide and reactive oxygen species, Free Radio. Biol. Med. 48(2010) 905-914.

[50] Z. Xun, D. Y. Lee, J. Lim, C. A. Canaria, A. Barnebey, S. M. Yanonne, C. T. McMurray, Retinoic acid-induced differentiation increases the rate of oxygen consumption and enhances the spare respiratory capacity of mitochondria in SH-SY5Y cells, Mech. Ageing Dev. 133 (2012) 176-185.

[51] L. Schneider, S. Giordano, B. R. Zelickson, S. Johnson, A. Benavides, X. Ouyang, N. Fineberg, V. M. Darley-Usmar, J. Zhang, Differentiation of SH-SY5Y cells to a neuronal phenotype changes cellular bioenergetics and the response to oxidative stress, Free Radio. Biol. Med. 51 (2011) 2007-2017.

[52] A. S. Divakaruni, M. D. Brand, The regulation and physiology of mitochondrial proton leak, Physiology. (Bethesda.) 26 (2011) 192-205.

[53] S. Giordano, J. Lee, V. M. Darley-Usmar, J. Zhang, Distinct effects of rotenone, 1-methyl-4-phenylpyridinium and 6-hydroxydopamine on cellular bioenergetics and cell death, PLoS. One. 7 (2012) e44610.

[54] J. Lee, C. H. Kim, D. K. Simon, L. R. Aminova, A. Y. Andreyev, Y. E. Kushnareva, A. N. Murphy, B. E. Lonze, K. S. Kim, D. D. Ginty, R. J. Ferrante, H. Ryu, R. R. Ratan, Mitochondrial cyclic AMP response element-binding protein (CREB) mediates mitochondrial gene expression and neuronal survival, J. Biol. Chem. 280 (2005) 40398-40401.

[55] M. D. Brand, D. G. Nicholls, Assessing mitochondrial dysfunction in cells, Biochem. J. 435 (2011) 297-312.

[56] O. Cooper, H. Seo, S. Andrabi, C. Guardia-Laguarta, J. Graziotto, M. Sundberg, J. R. McLean, L. Carrillo-Reid, Z. Xie, T. Osborn, G. Hargus, M. Deleidi, T. Lawson, H. Bogetofte, E. Perez-Torres, L. Clark, C. Moskowitz, J. Mazzulli, L. Chen, L. Volpicelli-Daley, N. Romerb, H. Jiang, R. J. Uitti, Z. Huang, G. Opala, L. A. Scarffe, V. L. Dawson, C. Klein, J. Feng, O. A. Ross, J. Q. Trojanowski, V. M. Lee, K. Marder, D. J. Surmeier, Z. K. Wszolek, S. Przedborski, D. Krainc, T. M. Dawson, O. Isacson, Pharmacological rescue of mitochondrial deficits in iPSC-derived neural cells from patients with familial Parkinson's disease, Sci. Transl. Med. 4 (2012) 141ra90.

[57] Z. A. Shah, R. C. Li, A. S. Ahmad, T. W. Kensler, M. Yamamoto, S. Biswal, S. Dore, The flavanol (−)-epicatechin prevents stroke damage through the Nrf2/HO1 pathway, J. Cereb. Blood Flow Metab 30 (2010) 1951-1961.

[58] A. Ahmad, M. M. Khan, M. N. Hoda, S. S. Raza, M. B. Khan, H. Javed, T. Ishrat, M. Ashafaq, M. E. Ahnnad, M. M. Safhl, F. Islam, Quercetin protects against oxidative stress associated damages in a rat model of transient focal cerebral ischemia and reperfusion, Neurochem. Res. 36 (2011) 1360-1371.

[59] J. K. Lee, H. J. Kwak, M. S. Piao, J. W. Jang, S. H. Kinn, H. S. Kim, Quercetin reduces the elevated matrix metalloproteinases-9 level and improves functional outcome after cerebral focal ischemia in rats, Acta Neurochir. (Wien.) 153 (2011) 1321-1329.

[60] C. H. Kim, S. U. Kang, J. Pyun, M. H. Lee, H. S. Hwang, H. Lee, Epicatechin protects auditory cells against cisplatin-induced death, Apoptosis. 13 (2008) 1184-1194.

[61] J. S. Lee, S. U. Kang, H. S. Hwang, J. H. Pyun, Y. H. Choung, C. H. Kim, Epicatechin protects the auditory organ by attenuating cisplatin-induced ototoxicity through inhibition of ERK, Toxicol. Lett. 199 (2010) 308-316.

[62] P. Pignatelli, S. S. Di, B.Buchetti, V. Sanguigni, A. Brunelli, F. Violi, Polyphenols enhance platelet nitric oxide by inhibiting protein kinase C-dependent NADPH oxidase activation: effect on platelet recruitment, FASEB J. 20 (2006) 1082-1089.

[63] M. H. Jeong, J. S. Kim, Y. Zou, C. S. Yoon, H. W. Lim, J. Ahn, H. Y. Lee, Enhancement of pheochromocytoma nerve cell growth by consecutive fractionization of Angelica gigas Nakai extracts, Cytotechnology 62 (2010) 461-472.

[64] J. Y. Jeng, W. H. Lee, Y. H. Tsai, C. Y. Chen, S. Y. Chao, R. H. Hsieh, Functional modulation of mitochondria by eicosapentaenoic acid provides protection against ceramide toxicity to C6 glioma cells, J. Agric. Food Chem. 57 (2009) 11455-11462.

[65] S. Kamisli, O. Ciftci, A. Cetin, K. Kaya, O. Kannisli, H. Celik, Fish oil protects the peripheral and central nervous systems against cisplatin-induced neurotoxicity, Nutr. Neurosci. (2013).

[66] B. Gopinath, V. M. Flood, E. Rochtchina, C. M. McMahon, P. Mitchell, Consumption of omega-3 fatty acids and fish and risk of age-related hearing loss, Am. J. Clin. Nutr. 92 (2010) 416-421.

[67] K. M. Denny Joseph, Muralidhara, Enhanced neuroprotective effect of fish oil in combination with quercetin against 3-nitropropionic acid induced oxidative stress in rat brain, Prog. Neuropsychopharmacol. Biol. Psychiatry 40 (2013) 83-92.

[68] B. Giunta, H. Hou, Y. Zhu, J. Salemi, A. Ruscin, R. D. Shytle, J. Tan, Fish oil enhances anti-amyloidogenic properties of green tea EGCG in Tg2576 mice, Neurosci. Lett. 471 (2010) 134-138.

[69] J. A. Ross, C. M. Kasum, Dietary flavonoids: bioavailability, metabolic effects, and safety, Annu. Rev. Nutr. 22 (2002) 19-34.

[70] C. M. Ballantyne, H. E. Bays, J. J. Kastelein, E. Stein, J. L. Isaacsohn, R. A. Braeckman, P. N. Soni, Efficacy and safety of eicosapentaenoic acid ethyl ester (AMR101) therapy in statin-treated patients with persistent high triglycerides (from the ANCHOR study), Am. J. Cardiol. 110 (2012) 984-992.

[71] C. I. Janssen, A. J. Kiliaan, Long-chain polyunsaturated fatty acids (LCPUFA) from genesis to senescence: The influence of LCPUFA on neural development, aging, and neurodegeneration, Prog. Lipid Res. 53C (2013) 1-17.

[72] X. Gao, A. Cassidy, M. A. Schwarzschild, E. B. Rimm, A. Ascherio, Habitual intake of dietary flavonoids and risk of Parkinson disease, Neurology 78 (2012) 1138-1145.

[73] F. Kamel, S. M. Goldnnan, D. M. Umbach, H. Chen, G. Richardson, M. R. Barber, C. Meng, C. Marras, M. Korell, M. Kasten, J. A. Hoppin, K. Comyns, A. Chade, A. Blair, G. S. Bhudhikanok, R. G. Webster, L. J. William, D. P. Sandler, C. M. Tanner, Dietary fat intake, pesticide use, and Parkinson's disease, Parkinsonism. Relat Disord. (2013).

[74] A. Cassidy, E. B. Rimm, E. J. O'Reilly, G. Logroscino, C. Kay, S. E. Chiuve, K. M. Rexrode, Dietary Flavonoids and Risk of Stroke in Women, Stroke (2012).

[75] E. J. Feskens, D. Kromhout, Epidemiologic studies on Eskimos and fish intake, Ann. N. Y. Acad. Sci. 683 (1993) 9-15.

[76] K. Beking, A. Vieira, Flavonoid intake and disability-adjusted life years due to Alzheimer's and related dementias: a population-based study involving twenty-three developed countries, Public Health Nutr. 13 (2010) 1403-1409.

[77] P. Barberger-Gateau, C. Raffaitin, L. Letenneur, C. Berr, C. Tzourio, J. F. Dartigues, A. Alperovitch, Dietary patterns and risk of dementia: the Three-City cohort study, Neurology 69 (2007) 1921-1930.

[78] T. H. Murphy, L. A. Blatter, W. G. Wier, J. M. Baraban, Spontaneous synchronous synaptic calcium transients in cultured cortical neurons, J. Neurosci. 12 (1992) 4834-4845.

[79] M. A. Dichter, Rat cortical neurons in cell culture: culture methods, cell morphology, electrophysiology, and synapse formation, Brain Res. 149 (1978) 279-293.

[80] C. H. Lin, P. S. Chen, P. W. Gean, Glutamate preconditioning prevents neuronal death induced by combined oxygen-glucose deprivation in cultured cortical neurons, Eur. J. Pharmacol. 589 (2008) 85-93.

[81] R. Tremblay, B. Chakravarthy, K. Hewitt, J. Tauskela, P. Morley, T. Atkinson, J. P. Durkin, Transient NMDA receptor inactivation provides long-term protection to cultured cortical neurons from a variety of death signals, J. Neurosci. 20 (2000) 7183-7192.

[82] J. S. Tauskela, H. Fang, M. Hewitt, E. Brunette, T. Ahuja, J. P. Thivierge, T. Comas, G. A. Mealing, Elevated synaptic activity preconditions neurons against an in vitro model of ischemia, J. Biol. Chem. 283 (2008) 34667-34676.

[83] M. Montero, C. D. Lobaton, E. Hernandez-Sanmiguel, J. Santodomingo, L. Vay, A. Moreno, J. Alvarez, Direct activation of the mitochondrial calcium uniporter by natural plant flavonoids, Biochem. J. 384 (2004) 19-24.

[84] R. M. Denton, Regulation of mitochondrial dehydrogenases by calcium ions, Biochim. Biophys. Acta 1787 (2009) 1309-1316.

[85] J. Satrustegui, B. Pardo, A. A. del, Mitochondrial transporters as novel targets for intracellular calcium signaling, Physiol Rev. 87 (2007) 29-67.

[86] P. R. Territo, V. K. Mootha, S. A. French, R. S. Balaban, Ca(2+) activation of heart mitochondrial oxidative phosphorylation: role of the F(0)/F(1)-ATPase, Am. J. Physiol Cell Physiol 278 (2000) C423-C435.

[87] Z. Xun, S. Rivera-Sanchez, S. Ayala-Pena, J. Lim, H. Budworth, E. M. Skoda, P. D. Robbins, L. J. Niedernhofer, P. Wipf, C. T. McMurray, Targeting of XJB-5-131 to mitochondria suppresses oxidative DNA damage and motor decline in a mouse model of Huntington's disease, Cell Rep. 2 (2012) 1137-1142.

[88] J. C. Corona, S. C. de Souza, M. R. Duchen, PPARgamma activation rescues mitochondriat function from inhibition of complex I and loss of PINK1, Exp. Neural. 253 (2014) 16-27.

[89] R. Acin-Perez, E. Salazar, M. Kamenetsky, J. Buck, L. R. Levin, G. Manfredi, Cyclic AMP produced inside mitochondria regulates oxidative phosphorylation, Cell Metab 9 (2009) 265-276.

[90] A. M. Sardanelli, Z. Technikova-Dobrova, F. Speranza, A. Mazzocca, S. Scacco, S. Papa, Topology of the mitochondrial cAMP-dependent protein kinase and its substrates, FEBS Lett. 396 (1996) 276-278.

[91] B. G. Di, E. Scalzotto, M. Mongillo, T. Pozzan, Mitochondrial Ca(2)(+) uptake induces cyclic AMP generation in the matrix and modulates organelle ATP levels, Cell Metab 17 (2013) 965-975.

[92] M. Fiorani, A. Guidarelli, M. Blasa, C. Azzolini, M. Candiracci, E. Piatti, O. Cantoni, Mitochondria accumulate large amounts of quercetin: prevention of mitochondrial damage and release upon oxidation of the extramitochondrial fraction of the flavonoid, J. Nutr. Biochem. 21(2010) 397-404.

[93] E. K. Schroeder, N. A. Kelsey, J. Doyle, E. Breed, R. J. Bouchard, F. A. Loucks, R. A. Harbison, D. A. Linseman, Green tea epigallocatechin 3-gallate accumulates in mitochondria and displays a selective antiapoptotic effect against inducers of mitochondrial oxidative stress in neurons, Antioxid. Redox. Signal. 11(2009) 469-480.

[94] W. C. Ko, C. M. Shih, Y. H. Lai, J. H. Chen, H. L. Huang, Inhibitory effects of flavonoids on phosphodiesterase isozymes from guinea pig and their structure-activity relationships, Biochem. Pharmacol. 68 (2004) 2087-2094.

[95] M. R. Peluso, Flavonoids attenuate cardiovascular disease, inhibit phosphodiesterase, and modulate lipid homeostasis in adipose tissue and liver, Exp. Biol. Med. (Maywood.) 231 (2006) 1287-1299.

[96] E. Alvarez, M. Campos-Toimil, H. Justiniano-Basaran, C. Lugnier, F. Orallo, Study of the mechanisms involved in the vasorelaxation induced by (−)-epigallocatechin-3-gallate in rat aorta, Br. J. Pharmacol. 147 (2006) 269-280.

[97] R. Acin-Perez, M. Russwurm, K. Gunnewig, M. Gertz, G. Zoidl, L. Ramos, J. Buck, L. R. Levin, J. Rassow, G. Manfredi, C. Steegborn, A phosphodiesterase 2A isoform localized to mitochondria regulates respiration, J. Biol. Chem. 286 (2011) 30423-30432.

[98] J. St-Pierre, S. Drori, M. Uldry, J. M. Silvaggi, J. Rhee, S. Jager, C. Handschin, K. Zheng, J. Lin, W. Yang, D. K. Simon, R. Bachoo, B. M. Spiegelman, Suppression of reactive oxygen species and neurodegeneration by the PGC-1 transcriptional coactivators, Cell 127 (2006) 397-408.

[99] P. Wareski, A. Vaarmann, V. Choubey, D. Safiulina, J. Liiv, M. Kuum, A. Kaasik, PGC-1{alpha} and PGC-1{beta} regulate mitochondrial density in neurons, J. Biol. Chem. 284 (2009) 21379-21385.

[100] N. Mendelev, S. L. Mehta, H. Idris, S. Kumari, P. A. Li, Selenite stimulates mitochondrial biogenesis signaling and enhances mitochondrial functional performance in murine hippocampal neuronal cells, PLoS. One. 7 (2012) e47910.

[101] S. L. Mehta, S. Kumari, N. Mendelev, P. A. Li, Selenium preserves mitochondrial function, stimulates mitochondrial biogenesis, and reduces infarct volume after focal cerebral ischemia, BMC. Neurosci. 13 (2012) 79.

[102] P. R. Brock, K. R. Knight, D. R. Freyer, K. C. Campbell, P. S. Steyger, B. W. Blakley, S. R. Rassekh, K. W. Chang, B. J. Fligor, K. Rajput, M. Sullivan, E. A. Neuwelt, Platinum-induced ototoxicity in children: a consensus review on mechanisms, predisposition, and protection, including a new International Society of Pediatric Oncology Boston ototoxicity scale, J. Clin. Oncol. 30 (2012) 2408-2417.

[103] S. S. More, O. Akil, A. G. lanculescu, E. G. Geier, L. R. Lustig, K. M. Giacomini, Role of the copper transporter, CTR1, in platinum-induced ototoxicity, J. Neurosci. 30 (2010) 9500-9509.

[104] L. Zhao, Q. Cheng, Z. Wang, Z. Xi, D. Xu, Y. Liu, Cisplatin binds to human copper chaperone Cox17: the mechanistic implication of drug delivery to mitochondria, Chem. Commun. (Camb.) 50 (2014) 2667-2669.

[105] R. Marullo, E. Werner, N. Degtyareva, B. Moore, G. Altavilla, S. S. Ramalingam, P. W. Doetsch, Cisplatin induces a mitochondrial-ROS response that contributes to cytotoxicity depending on mitochondrial redox status and bioenergetic functions, PLoS One 8 (2013) e81162.

[106] T. Langer, A. am Zehnhoff-Dinnesen, S. Radtke, J. Meitert, O. Zolk, Understanding platinum-induced ototoxicity, Trends Pharmacol. Sci. 34 (2013) 458-469.

[107] Y. Li, D. Ding, H. Jiang, Y. Fu, R. Salvi, Co-administration of cisplatin and furosemide causes rapid and massive loss of cochlear hair cells in mice, Neurotox. Res. 20 (2011) 307-319.

[108] E. J. Yang, G. S. Kim, J. A. Kim, K. S. Song, Protective effects of onion-derived quercetin on glutamate-mediated hippocampal neuronal cell death, Pharmacogn. Mag. 9 (2013) 302-308.

[109] B. Silva, P. J. Oliveira, A. Dias, J. O. Malva, Quercetin, kaempferol and biapigenin from Hypericum perforatum are neuroprotective against excitotoxic insults, Neurotox. Res. 13 (2008) 265-279.

[110] K. Ishige, D. Schubert, Y. Sagara, Flavonoids protect neuronal cells from oxidative stress by three distinct mechanisms, Free Radio. Biol. Med. 30 (2001) 433-446.

[111] R. Soundararajan, A. D. Wishart, H. P. Rupasinghe, M. Arcellana-Panlilio, C. M. Nelson, M. Mayne, G. S. Robertson, Quercetin 3-glucoside protects neuroblastoma (SH-SY5Y) cells in vitro against oxidative damage by inducing sterol regulatory element-binding protein-2-mediated cholesterol biosynthesis, J. Biol. Chem. 283 (2008) 2231-2246.

[112] C. Wagner, A. P. Vargas, D. H. Roos, A. F. Morel, M. Farina, C. W. Nogueira, M. Aschner, J. B. Rocha, Comparative study of quercetin and its two glycoside derivatives quercitrin and rutin against methylmercury (MeHg)-induced ROS production in rat brain slices, Arch. Toxicol. 84 (2010) 89-97.

[113] F. Roshanzamir, R. Yazdanparast, Quercetin attenuates cell apoptosis of oxidant-stressed SK-N-MC cells while suppressing up-regulation of the defensive element, HIF-1alpha, Neuroscience 277 (2014) 780-793.

[114] C. Wagner, R. Fachinetto, C. L. Dalla Corte, V. B. Brito, D. Severo, D. G. de Oliveira Costa, A. F. Morel, C. W. Nogueira, J. B. Rocha, Quercitrin, a glycoside form of quercetin, prevents lipid peroxidation in vitro, Brain Res. 1107 (2006) 192-198.

[115] S. S. Karuppagounder, S. K. Madathil, M. Pandey, R. Haobam, U. Rajamma, K. P. Mohanakumar, Quercetin up-regulates mitochondrial complex-I activity to protect against programmed cell death in rotenone model of Parkinson's disease in rats, Neuroscience 236 (2013) 136-148.

[116] G. Filomeni, I. Graziani, Z. D. De, L. Dini, D. Centonze, G. Rotilio, M. R. Ciriolo, Neuroprotection of kaennpferol by autophagy in models of rotenone-mediated acute toxicity: possible implications for Parkinson's disease, Neurobiol. Aging 33 (2012) 767-785.

[117] L. D. Mercer, B. L. Kelly, M. K. Horne, P. M. Beart, Dietary polyphenols protect dopamine neurons from oxidative insults and apoptosis: investigations in primary rat mesencephalic cultures, Biochem. Pharmacol. 69 (2005) 339-345.

[118] C. P. Wang, J. L. Li, L. Z. Zhang, X. C. Zhang, S. Yu, X. M. Liang, F. Ding, Z. W. Wang, lsoquercetin protects cortical neurons from oxygen-glucose deprivation-reperfusion induced injury via suppression of TLR4-NF-small ka, CyrillicB signal pathway, Neurochem. Int. 63 (2013) 741-749.

[119] O. Orban-Gyapai, A. Raghavan, A. Vasas, P. Forgo, J. Hohmann, Z. A. Shah, Flavonoids Isolated from Rumex aquaticus Exhibit Neuroprotective and Neurorestorative Properties by Enhancing Neurite Outgrowth and Synaptophysin, CNS. Neurol. Disord. Drug Targets. 13 (2014) 1458-1464.

[120] U. Gundimeda, T. H. McNeill, A. A. Elhiani, J. E. Schiffman, D. R. Hinton, R. Gopalakrishna, Green tea polyphenols precondition against cell death induced by oxygen-glucose deprivation via stimulation of laminin receptor, generation of reactive oxygen species, and activation of protein kinase Cepsilon, J. Biol. Chem. 287 (2012) 34694-34708.

[121] C. Manach, G. Williamson, C. Morand, A. Scalbert, C. Remesy, Bioavailability and bioefficacy of polyphenols in humans. I. Review of 97 bioavailability studies, Am. J. Clin. Nutr. 81(2005) 230S-242S.

[122] G. Williamson, C. Manach, Bioavailability and bioefficacy of polyphenols in humans. II. Review of 93 intervention studies, Am. J. Clin. Nutr. 81(2005) 2435-255S.

[123] M. M. Abd El Mohsen, G. Kuhnle, A. R. Rechner, H. Schroeter, S. Rose, P. Jenner, C. A. Rice-Evans, Uptake and metabolism of epicatechin and its access to the brain after oral ingestion, Free Radic. Biol. Med. 33 (2002) 1693-1702.

[124] L. Ho, M. G. Ferruzzi, E. M. Janle, J. Wang, B. Gong, T. Y. Chen, J. Lobo, B. Cooper, Q. L. Wu, S. T. Talcott, S. S. Percival, J. E. Simon, G. M. Pasinetti, Identification of brain-targeted bioactive dietary quercetin-3-O-glucuronide as a novel intervention for Alzheimer's disease, FASEB J. 27 (2013) 769-781.

[125] J. Wang, M. G. Ferruzzi, L. Ho, J. Blount, E. M. Janle, B. Gong, Y. Pan, G. A. Gowda, D. Raftery, I. Arrieta-Cruz, V. Sharma, B. Cooper, J. Lobo, J. E. Simon, C. Zhang, A. Cheng, X. Qian, K. Ono, D. B. Teplow, C. Pavlides, R. A. Dixon, G. M. Pasinetti, Brain-targeted proanthocyanidin metabolites for Alzheimer's disease treatment, J. Neurosci. 32 (2012) 5144-5150.

[126] J. Warford, Q. R. Jones, M. Nichols, V. Sullivan, H. P. Rupasinghe, G. S. Robertson, The flavonoid-enriched fraction AF4 suppresses neuroinflammation and promotes restorative gene expression in a mouse model of experimental autoimmune encephalomyelitis, J. Neuroimmunol. 268 (2014) 71-83.

[127] L. Rangel-Ordonez, M. Noldner, M. Schubert-Zsilavecz, M. Wurglics, Plasma levels and distribution of flavonoids in rat brain after single and repeated doses of standardized Ginkgo biloba extract EGb 761(R), Planta Med. 76 (2010) 1683-1690.

[128] B. Ossola, T. M. Kaariainen, P. T. Mannisto, The multiple faces of quercetin in neuroprotection, Expert. Opin. Drug Saf 8 (2009) 397-409.

[129] A. Scalbert, C. Manach, C. Morand, C. Remesy, L. Jimenez, Dietary polyphenols and the prevention of diseases, Grit Rev. Food Sci. Nutr. 45 (2005) 287-306.

[130] J. P.Spencer, The interactions of flavonoids within neuronal signalling pathways, Genes Nutr. 2 (2007) 257-273.

[131] S. M. Dravid, T. F. Murray, Spontaneous synchronized calcium oscillations in neocortical neurons in the presence of physiological [Mg(2+)]: involvement of AMPA/kainate and metabotropic glutamate receptors, Brain Res. 1006 (2004) 8-17.

[132] T. Opitz, A. D. De Lima, T. Voigt, Spontaneous development of synchronous oscillatory activity during maturation of cortical networks in vitro, J. Neurophysiol. 88 (2002) 2196-2206.

[133] K. W. Young, E. T. Bampton, L. Pinon, D. Bano, P. Nicotera, Mitochondrial Ca2+ signalling in hippocampal neurons, Cell Calcium 43 (2008) 296-306.

[134] B. Billups, I. D. Forsythe, Presynaptic mitochondrial calcium sequestration influences transmission at mammalian central synapses, J. Neurosci. 22 (2002) 5840-5847.

[135] O. Kann, R. Kovacs, Mitochondria and neuronal activity, Am. J. Physiol Cell Physiol 292 (2007) C641-C657.

[136] I. Llorente-Folch, C. B. Rueda, I. Amigo, A. A. del, T. Saheki, B. Pardo, J. Satrustegui, Calcium-regulation of mitochondrial respiration maintains ATP homeostasis and requires ARALAR/AGC1-malate aspartate shuttle in intact cortical neurons, J. Neurosci. 33 (2013) 13957-71, 13971a.

[137] A. K. Chouhan, M. V. Ivannikov, Z. Lu, M. Sugimori, R. R. Llinas, G. T. Macleod, Cytosolic calcium coordinates mitochondrial energy metabolism with presynaptic activity, J. Neurosci. 32 (2012) 1233-1243.

[138] J. Qiu, Y. W. Tan, A. M. Hagenston, M. A. Martel, N. Kneisel, P. A. Skehel, D. J. Wyllie, H. Bading, G. E. Hardingham, Mitochondrial calcium uniporter Mcu controls excitotoxicity and is transcriptionally repressed by neuroprotective nuclear calcium signals, Nat. Commun. 4 (2013) 2034.

[139] M. Patron, A. Raffaello, V. Granatiero, A. Tosatto, G. Merli, S. D. De, L. Wright, G. Pallafacchina, A. Terrin, C. Mammucari, R. Rizzuto, The mitochondrial calcium uniporter (MCU): molecular identity and physiological roles, J. Biol. Chem. 288 (2013) 10750-10758.

[140] G. P. Sergeant, E. Bradley, K. D. Thornbury, N. G. McHale, M. A. Hollywood, Role of mitochondria in modulation of spontaneous Ca2+ waves in freshly dispersed interstitial cells of Cajal from the rabbit urethra, J. Physiol 586 (2008) 4631-4642.

[141] B. G. Sanganahalli, P. Herman, F. Hyder, S. S. Kannurpatti, Mitochondrial functional state impacts spontaneous neocortical activity and resting state FMRI, PLoS One 8 (2013) e63317.

[142] B. G. Sanganahalli, P. Herman, F. Hyder, S. S. Kannurpatti, Mitochondrial calcium uptake capacity modulates neocortical excitability, J. Cereb. Blood Flow Metab 33 (2013) 1115-1126.

[143] J. H. Schellens, A. S. Planting, J. Ma, M. Maliepaard, V. A. de, D. M. de Boer, J. Verweij, Adaptive intrapatient dose escalation of cisplatin in patients with advanced head and neck cancer, Anticancer Drugs 12 (2001) 667-675.

[144] A. L. Poirrier, P. Van Den Ackerveken, T. S. Kim, R. Vandenbosch, L. Nguyen, P. P. Lefebvre, B. Malgrange, Ototoxic drugs: difference in sensitivity between mice and guinea pigs, Toxicol. Lett. 193 (2010) 41-49.

[145] J. E. Rice, III, R. C. Vannucci, J. B. Brierley, The influence of immaturity on hypoxic-ischemic brain damage in the rat, Ann: Neurol. 9 (1981) 131-141.

[146] F. Adhami, G. Liao, Y. M. Morozov, A. Schloemer, V. J. Schmithorst, J. N. Lorenz, R. S. Dunn, C. V. Vorhees, M. Wills-Karp, J. L. Degen, R. J. Davis, N. Mizushima, P. Rakic, B. J. Dardzinski, S. K. Holland, F. R. Sharp, C. Y. Kuan, Cerebral ischemia-hypoxia induces intravascular coagulation and autophagy, Am. J. Pathol. 169 (2006) 566-583.

[147] E. E. Olson, R. J. McKeon, Characterization of cellular and neurological damage following unilateral hypoxia/ischemia, J. Neurol. Sci. 227 (2004) 7-19.

[148] C. D. Cowper-Smith, G. J. Anger, E. Magal, M. H. Norman, G. S. Robertson, Delayed administration of a potent cyan dependent kinase and glycogen synthase kinase 3 beta inhibitor produces long-term neuroprotection in a hypoxia-ischemia model of brain injury, Neuroscience 155 (2008) 864-875.

[149] B. H. Han, D. Xu, J. Choi, Y. Han, S. Xanthoudakis, S. Roy, J. Tann, J. Vaillancourt, J. Colucci, R. Siman, A. Giroux, G. S. Robertson, R. Zannboni, D. W. Nicholson, D. M. Holtzman, Selective, reversible caspase-3 inhibitor is neuroprotective and reveals distinct pathways of cell death after neonatal hypoxic-ischemic brain injury, J. Biol. Chem. 277 (2002) 30128-30136.

[150] S. Mathai, A. J. Gunn, R. A. Backhaus, J. Guan, Window of opportunity for neuroprotection with an antioxidant, allene oxide synthase, after hypoxia-ischemia in adult male rats, CNS. Neurosci. Ther. 18 (2012) 887-894.

[151] J. Guan, S. Mathai, P. Harris, J. Y. Wen, R. Zhang, M. Brimble, P. Gluckman, Peripheral administration of a novel diketopiperazine, NNZ 2591, prevents brain injury and improves somatosensory-motor function following hypoxia-ischemia in adult rats, Neuropharmacology 53 (2007) 749-762.

[152] B. J. Schaller, Influence of age on stroke and preconditioning-induced ischemic tolerance in the brain, Exp. Neurol. 205 (2007) 9-19.

[153] P. G. Keddy, K. Dunlop, J. Warford, M. L. Samson, Q. R. Jones, H. P. Rupasinghe, G. S. Robertson, Neuroprotective and anti-inflammatory effects of the flavonoid-enriched fraction AF4 in a mouse model of hypoxic-ischemic brain injury, PLoS. One. 7 (2012) e51324.

[154] T. M. Kovalenko, I. O. Osadchenko, O. M. Tsupykov, T. A. Pivneva, A. S. Shalamai, O. O. Moibenko, H. H. Skybo, [Neuroprotective effect of quercetin during experimental brain ischemia], Fiziol. Zh. 52 (2006) 21-27.

[155] F. Pu, K. Mishima, K. Irie, K. Motohashi, Y. Tanaka, K. Orito, T. Egawa, Y. Kitamura, N. Egashira, K. Iwasaki, M. Fujiwara, Neuroprotective effects of quercetin and rutin on spatial memory impairment in an 8-arm radial maze task and neuronal death induced by repeated cerebral ischemia in rats, J. Pharmacol. Sci. 104 (2007) 329-334.

[156] J. Katchanov, C. Harms, K. Gertz, L. Hauck, C. Waeber, L. Hirt, J. Priller, H. R. von, W. Bruck, H. Hortnagl, U. Dirnagl, P. G. Bhide, M. Endres, Mild cerebral ischemia induces loss of cyclin-dependent kinase inhibitors and activation of cell cycle machinery before delayed neuronal cell death, J. Neurosci. 21(2001) 5045-5053.

[157] K. J. Livak, T. D. Schmittgen, Analysis of relative gene expression data using real-time quantitative PCR and the 2(-Delta Delta C(T)) Method, Methods 25 (2001) 402-408.

1. A method for treating, prophylactically treating, preventing or reducing the severity of oxidative injury resulting from mitochondrial dysfunction comprising administering to an individual in need of such treatment an effective amount of a composition comprising a flavan-3-ol, a flavonoid and a fatty acid.

2. The method according to claim 1 wherein the oxidative injury resulting from mitochondrial dysfunction is a neurodegenerative disorder.

3. The method according to claim 1 wherein the oxidative injury resulting from mitochondrial dysfunction is ototoxicity.

4. The method according to claim 2 wherein the neurodegenerative disorder is selected from the group consisting of Parkinson's disease; stroke; Huntington's disease; amyotrphic lateral sclerosis (ALS); Alzheimer's disease; and Multiple Sclerosis (MS).

5. The method according to claim 1 wherein the flavonoid is selected from the group consisting of Quercetin; lsorhamnetin (3-Methylquercetin, 3,5,7-trihydroxy-2-(4-hydroxy-3-methoxyphenyl)chromen-4-one); Rutin (Quercetin-3-O-rutinoside, 2-(3,4-dihydroxyphenyl)-5,7-dihydroxy-3-[α-L-rhamnopyranosyl-(1→6)-3-D-glucopyranosyloxy]-4H-chromen-4-one); Quercitrin (Quercetin 3-O-rhamnoside, 2-(3,4-Dihydroxyphenyl)-5,7-dihydroxy-3-[[(2S,3R,4R,5R,6S)-3,4,5-trihydroxy-6-methyl-2-tetrahydropyranyl]oxy]-4-chrornenone); Hyperoside (Quercetin-3-O-galactoside, 2-(3,4-dihydroxyphenyl)-3-[(3R,4S,5R,6R)-3,4,5-trihydroxy-6-(hydroxymethyl)oxan-2-yl]oxy-4H-chromene-4,5,7-triol); Taxifolin (Dihydroquercetin, (2R,3R)-2-(3,4-dihydroxyphenyl)-3,5,7-trihydroxy-2, 3-dihydrochromen-4-one); Querectin-3-O-glucoside; Quercetin-3-O-maltoside; Quercetin-3-O-gentiobioside; α-monoglucosyl rutin; α-oligoglucosyl rutin; α-oligoglucosyl isoquercitrin (enzymatically modified isoquercitrin, EMIQ); Apigenin (5,7-Dihydroxy-2-(4-hydroxyphenyl)-4H-1-benzopyran-4-one); Homoeriodictyol ((2S)-5,7-Dihydroxy-2-(4-hydroxy-3-methoxyphenyl)-4-chromanone); Galangin (3,5,7-trihydroxy-2-phenylchromen-4-one); Myricetin (3,5,7-Trihydroxy-2-(3,4,5-trihydroxyphenyl)-4-chromenone; Kaempferol (3,5,7-Trihydroxy-2-(4-hydroxyphenyl)-4H-chromen-4-one); Aromadendrin (Dihydrokaempferol, (2R,3R)-3,5,7-trihydroxy-2-(4-hydroxyphenyl)-2,3-dihydrochromen-4-one); Pachypodol (5-hydroxy-2-(4-hydroxy-3-methoxyphenyl)-3,7-dimethoxychromen-4-one); Fisetin (2-(3,4-dihydroxyphenyl)-3,7-dihydroxychromen-4-one); Hesperetin ((S)-2,3-dihydro-5,7-dihydroxy-2-(3-hydroxy-4-methoxyphenyl)-4H-1-benzopyran-4-one); Hesperidin (Hesperetin 7-rutinoside); Baicalein (5,6,7-Trihydroxy-2-phenyl-chromen-4-one); Baicalin (Baicalein 7-O-glucuronide); Tetuin (Baicalein 6-O-glucoside); Luteolin (2-(3,4-Dihydroxyphenyl)-5,7-dihydroxy-4-chromenone); Cynaroside (Luteolin 7-O-glucoside); Isoorientin (Luteolin 6-C-glucoside); Orientin (Luteolin 8-C-glucoside); Veronicastroside (Luteolin 7-O-neohesperidoside); Luteolin-7-O-glucuronide; Tangeritin (Tangeretin, 5,6,7,8-tetramethoxy-2-(4-methoxyphenyl)-4H-1-benzopyran-4-one); Giraldiin A (4′,5,5′,7-Tetrahydroxy-3-methoxy-3′-O-α-L-arabinopyranosyloxyflavone); Giraldiin B (5,5′,7′-Trihydroxy-2′,3-dimethoxy-4′-O-β-D-glucopyranosyloxyflavone); Naringenin (5,7-dihydroxy-2-(4-hydroxyphenyl)chroman-4-one); Nepetin (2-(3,4-dihydroxyphenyl)-5,7-dihydroxy-6-methoxychromen-4-one); Nepitrin (Nepetin 7-O-glucoside); Rhamnazin (3,5-dihydroxy-2-(4-hydroxy-3-methoxyphenyl)-7-methoxychromen-4-one) Wogonin (5,7-Dihydroxy-8-methoxy-2-phenyl-4H-chromen-4-one); and Scutellarin (7-(β-D-glucopyranuronosyloxy)-5,6-dihydroxy-2-(4-hydroxyphenyl)-4H-1-benzopyran-4-one).

6. The method according to claim 1 wherein the flavan-3-ol is selected from the group consisting of: (−)-Epicatechin; (+)-Catechin ((2R,3S)-2-(3,4-dihydroxyphenyl)-3,4-dihydro-2H-chromene-3,5,7-triol); (−)-Catechin ((2R,3R)-2-(3,4-dihydroxyphenyl)-3,4-dihydro-2H-chromene-3,5,7-triol); (+)-Epicatechin (2S,3S) ((2S,3S)-2-(3,4-dihydroxyphenyl)-3,4-dihydro-2H-chromene-3,5,7-triol); Gallocatechin ((2R,3R)-2-(3,4,5-trihydroxyphenyl)-3,4-dihydro-2H-benzopyran-3,5,7-triol); and Epigallocatechin gallate (EGCG) [(2R,3R)-5,7-dihydroxy-2-(3,4,5-trihydroxyphenyl)chroman-3-yl] 3,4,5-trihydroxybenzoate.

7. The method according to claim 1 wherein the fatty acid is selected from the group consisting of: EPA ethyl ester; Eicosapentaenoic acid (EPA (5Z,8Z,11Z,14Z,17Z)-5,8,11,14,17-icosapentaenoic acid); Docosahexaenoic acid ((4Z,7Z,10Z,13Z,16Z,19Z)-docosa-4,7,10,13,16,19-hexaenoic acid); α-Linolenic acid ((9Z,12Z,15Z)-9,12,15-Octadecatrienoic acid); γ-Linolenic acid (all-cis-6,9,12-octadecatrienoic acid); Linoleic acid ((9Z,12Z)-9,12-Octadecadienoic acid); Lipoic acid ((R)-5-(1,2-dithiolan-3-yl)pentanoic acid); Oleic acid ((9Z)-Octadec-9-enoic acid); Palmitoleic acid (hexadec-9-enoic acid); Vaccenic acid ((E)-Octadec-11-enoic acid); Rumenic acid ((9Z,11E)-Octadeca-9,11-dienoic acid); Hexadecatrienoic acid (HTA) (all-cis 7,10,13-hexadecatrienoic acid); Stearidonic acid (SDA) (all-cis-6,9,12,15,-octadecatetraenoic acid); Eicosatrienoic acid (ETE) (all-cis-11,14,17-eicosatrienoic acid); Eicosatetraenoic acid (ETA) (all-cis-8,11,14,17-eicosatetraenoic acid); Heneicosapentaenoic acid (HPA) (all-cis-6,9,12,15,18-heneicosapentaenoic acid); Docosapentaenoic acid (DPA, Clupanodonic acid (all-cis-7,10,13,16,19-docosapentaenoic acid)); Tetracosapentaenoic acid (all-cis-9,12,15,18,21-tetracosapentaenoic acid); Tetracosahexaenoic acid (Nisinic acid (all-cis-6,9,12,15,18,21-tetracosahexaenoic acid)); Eicosadienoic acid (all-cis-11,14-eicosadienoic acid); Dihomo-gamma-linolenic acid (DGLA (all-cis-8,11,14-eicosatrienoic acid)); Arachidonic acid (all-cis-5,8,11,14-eicosatetraenoic acid); Docosadienoic acid (all-cis-13,16-docosadienoic acid); Adrenic acid (all-cis-7,10,13,16-docosatetraenoic acid); Docosapentaenoic acid (Osbond acid (all-cis-4,7,10,13,16-docosapentaenoic acid)); Tetracosatetraenoic acid (all-cis-9,12,15,18-tetracosatetraenoic acid); Tetracosapentaenoic acid (all-cis-6,9,12,15,18-tetracosapentaenoic acid); Eicosenoic acid (cis-11-eicosenoic acid); Mead acid (all-cis-5,8,11-eicosatrienoic acid); Erucic acid (cis-13-docosenoic acid); Nervonic acid (cis-15-tetracosenoic acid); α-Calendic acid (8E,10E,12Z-octadecatrienoic acid); 3-Calendic acid (8E,10E,12E-octadecatrienoic acid); Jacaric acid (8Z,10E,12Z-octadecatrienoic acid); α-Eleostearic acid (9Z,11E,13E-octadeca-9,11,13-trienoic acid); 3-Eleostearic acid (9E,11E,13E-octadeca-9,11,13-trienoic acid); Catalpic acid (9Z,11Z,13E-octadeca-9,11,13-trienoic acid); Punicic acid (9Z,11E,13Z-octadeca-9,11,13-trienoic acid); Rumelenic acid (9E, 11Z,15E-octadeca-9,11,15-trienoic acid); α-Parinaric acid (9E,11Z,13Z,15E-octadeca-9,11,13,15-tetraenoic acid); 3-Parinaric acid (all trans-octadeca-9,11,13,15-tretraenoic acid); Bosseopentaenoic acid (5Z,8Z,10E,12E,14Z-eicosanoic acid); Pinolenic acid ((5Z,9Z,12Z)-octadeca-5,9,12-trienoic acid); vitamin E (α-Tocopherol, γ-Tocopherol), Podocarpic acid ((5Z,11Z,14Z)-eicosa-5,11,14-trienoic acid) and a plasmalogen.

8. The method according to claim 1 wherein the flavan-3-ol is (−)-Epicatechin, the flavonoid is Quercetin and the fatty acid is EPA ethyl ester.

9. The method according to claim 8 wherein the composition comprises 10-250 mg epicatechin and 10-250 mg quercetin suspended in 0.1-1 ml of eicosapentaenoic acid ethyl ester.

10. A composition for treating, prophylactically treating, preventing or reducing the severity of oxidative injury resulting from mitochondrial dysfunction comprising administering to an individual in need of such treatment an effective amount of a composition comprising a flavan-3-ol, a flavonoid and a fatty acid.

11. The composition according to claim 10 wherein the oxidative injury resulting from mitochondrial dysfunction is a neurodegenerative disorder.

12. The composition according to claim 10 wherein the oxidative injury resulting from mitochondria! dysfunction is ototoxicity.

13. The composition according to claim 11 wherein the neurodegenerative disorder is selected from the group consisting of Parkinson's disease; stroke; Huntington's disease; amyotrphic lateral sclerosis (ALS); Alzheimer's disease; and Multiple Sclerosis (MS).

14. The composition according to claim 10 wherein the flavonoid is selected from the group consisting of Quercetin; Isorhamnetin (3-Methylquercetin, 3,5,7-trihydroxy-2-(4-hydroxy-3-methoxyphenyl)chromen-4-one); Rutin (Quercetin-3-O-rutinoside, 2-(3,4-dihydroxyphenyl)-5,7-dihydroxy-3-[α-L-rhamnopyranosyl-(1→6)-β-D-glucopyranosyloxy]-4H-chromen-4-one); Quercitrin (Quercetin 3-O-rhamnoside, 2-(3,4-Dihydroxyphenyl)-5,7-dihydroxy-3-[[(2S,3R,4R,5R,6S)-3,4,5-trihydroxy-6-methyl-2-tetrahydropyranyl]oxy]-4-chromenone); Hyperoside (Quercetin-3-O-galactoside, 2-(3,4-dihydroxyphenyl)-3-[(3R,4S,5R,6R)-3,4,5-trihydroxy-6-(hydroxymethypoxan-2-yl]oxy-4H-chromene-4,5,7-triol); Taxifolin (Dihydroquercetin, (2R,3R)-2-(3,4-dihydroxyphenyl)-3,5,7-trihydroxy-2, 3-dihydrochromen-4-one); Querectin-3-O-glucoside; Quercetin-3-O-maltoside; Quercetin-3-O-gentiobioside; σ-monoglucosyl rutin; α-oligoglucosyl rutin; α-oligoglucosyl isoquercitrin (enzymatically modified isoquercitrin, EMIQ); Apigenin (5,7-Dihydroxy-2-(4-hydroxyphenyl)-4H-1-benzopyran-4-one); Homoeriodictyol ((2S)-5,7-Dihydroxy-2-(4-hydroxy-3-methoxyphenyl)-4-chromanone); Galangin (3,5,7-trihydroxy-2-phenylchromen-4-one); Myricetin (3,5,7-Trihydroxy-2-(3,4,5-trihydroxyphenyl)-4-chromenone; Kaempferol (3,5,7-Trihydroxy-2-(4-hydroxyphenyl)-4H-chromen-4-one); Aromadendrin (Dihydrokaempferol, (2R,3R)-3,5,7-trihydroxy-2-(4-hydroxyphenyl)-2,3-dihydrochromen-4-one); Pachypodol (5-hydroxy-2-(4-hydroxy-3-methoxyphenyl)-3,7-dimethoxychromen-4-one); Fisetin (2-(3,4-dihydroxyphenyl)-3,7-dihydroxychromen-4-one); Hesperetin ((S)-2,3-dihydro-5,7-dihydroxy-2-(3-hydroxy-4-methoxyphenyl)-4H-1-benzopyran-4-one); Hesperidin (Hesperetin 7-rutinoside); Baicalein (5,6,7-Trihydroxy-2-phenyl-chromen-4-one); Baicalin (Baicalein 7-O-glucuronide); Tetuin (Baicalein 6-O-glucoside); Luteolin (2-(3,4-Dihydroxyphenyl)-5,7-dihydroxy-4-chromenone); Cynaroside (Luteolin 7-O-glucoside); Isoorientin (Luteolin 6-C-glucoside); Orientin (Luteolin 8-C-glucoside); Veronicastroside (Luteolin 7-O-neohesperidoside); Luteolin-7-O-glucuronide; Tangeritin (Tangeretin, 5,6,7,8-tetramethoxy-2-(4-methoxyphenyl)-4H-1-benzopyran-4-one); Giraldiin A (4′,5,5′,7-Tetrahydroxy-3-methoxy-3′-O-α-L-arabinopyranosyloxyflavone); Giraldiin B (5,5′,7′-Trihydroxy-2′,3-dimethoxy-4′-O-β-D-glucopyranosyloxyflavone); Naringenin (5,7-dihydroxy-2-(4-hydroxyphenyl)chroman-4-one); Nepetin (2-(3,4-dihydroxyphenyl)-5,7-dihydroxy-6-methoxychromen-4-one); Nepitrin (Nepetin 7-O-glucoside); Rhannnazin (3,5-dihydroxy-2-(4-hydroxy-3-methoxyphenyl)-7-methoxychromen-4-one) Wagon in (5,7-Dihydroxy-8-Methoxy-2-phenyl-4H-chromen-4-one); and Scutellarin (7-(β-D-glucopyranuronosyloxy)-5,6-dihydroxy-2-(4-hydroxyphenyl)-4H-1-benzopyran-4-one).

15. The composition according to claim 10 wherein the flavan-3-ol is selected from the group consisting of: (−)-Epicatechin; (+)-Catechin ((2R,3S)-2-(3,4-dihydroxyphenyl)-3,4-dihydro-2H-chromene-3,5,7-triol); (−)-Catechin ((2R,3R)-2-(3,4-dihydroxyphenyl)-3,4-dihydro-2H-chromene-3,5,7-triol); (+)-Epicatechin (2S,3S) ((2S,3S)-2-(3,4-dihydroxyphenyl)-3,4-dihydro-2H-chromene-3,5,7-triol); Gallocatechin ((2R,3R)-2-(3,4,5-trihydroxyphenyl)-3,4-dihydro-2H-1-benzopyran-3,5,7-triol); and Epigallocatechin gallate (EGCG) [(2R,3R)-5,7-dihydroxy-2-(3,4,5-trihydroxyphenyl)chroman-3-yl] 3,4,5-trihydroxybenzoate.

16. The method according to claim 10 wherein the fatty acid is selected from the group consisting of: EPA ethyl ester; Eicosapentaenoic acid (EPA (5Z,8Z,11Z,14Z,17Z)-5,8,11,14,17-icosapentaenoic acid); Docosahexaenoic acid ((4Z,7Z,10Z,13Z,16Z,19Z)-docosa-4,7,10,13,16,19-hexaenoic acid); α-Linolenic acid ((9Z,12Z,15Z)-9,12,15-Octadecatrienoic acid); γ-Linolenic acid (all-cis-6,9,12-octadecatrienoic acid); Linoleic acid ((9Z,12Z)-9,12-Octadecadienoic acid); Lipoic acid ((R)-5-(1,2-dithiolan-3-yl)pentanoic acid); Oleic acid ((9Z)-Octadec-9-enoic acid); Palmitoleic acid (hexadec-9-enoic acid); Vaccenic acid ((E)-Octadec-11-enoic acid); Rumenic acid ((9Z,11E)-Octadeca-9,11-dienoic acid); Hexadecatrienoic acid (HTA) (all-cis 7,10,13-hexadecatrienoic acid); Stearidonic acid (SDA) (all-cis-6,9,12,15,-octadecatetraenoic acid); Eicosatrienoic acid (ETE) (all-cis-11,14,17-eicosatrienoic acid); Eicosatetraenoic acid (ETA) (all-cis-8,11,14,17-eicosatetraenoic acid); Heneicosapentaenoic acid (HPA) (all-cis-6,9,12,15,18-heneicosapentaenoic acid); Docosapentaenoic acid (DPA, Clupanodonic acid (all-cis-7,10,13,16,19-docosapentaenoic acid)); Tetra cosapentaenoic acid (all-cis-9,12,15,18,21-tetracosapentaenoic acid); Tetracosahexaenoic acid (Nisinic acid (all-cis-6,9,12,15,18,21-tetracosahexaenoic acid)); Eicosadienoic acid (all-cis-11,14-eicosadienoic acid); Dihomo-gamma-linolenic acid (DGLA (all-cis-8,11,14-eicosatrienoic acid)); Arachidonic acid (all-cis-5,8,11,14-eicosatetraenoic acid); Docosadienoic acid (all-cis-13,16-docosadienoic acid); Adrenic acid (all-cis-7,10,13,16-docosatetraenoic acid); Docosapentaenoic acid (Osbond acid (all-cis-4,7,10,13,16-docosapentaenoic acid)); Tetracosatetraenoic acid (all-cis-9,12,15,18-tetracosatetraenoic acid); Tetracosapentaenoic acid (all-cis-6,9,12,15,18-tetracosapentaenoic acid); Eicosenoic acid (cis-11-eicosenoic acid); Mead acid (all-cis-5,8,11-eicosatrienoic acid); Erucic acid (cis-13-docosenoic acid); Nervonic acid (cis-15-tetracosenoic acid); α-Calendic acid (8E,10E,12Z-octadecatrienoic acid); β-Calendic acid (8E,10E,12E-octadecatrienoic acid); Jacaric acid (8Z,10E,12Z-octadecatrienoic acid); α-Eleostearic acid (9Z,11E,13E-octadeca-9,11,13-trienoic acid); β-Eleostearic acid (9E,11E,13E-octadeca-9,11,13-trienoic acid); Catalpic acid (9Z,11Z,13E-octadeca-9,11,13-trienoic acid); Punicic acid (9Z,11E,13Z-octadeca-9,11,13-trienoic acid); Rumelenic acid (9E,11Z,15E-octadeca-9,11,15-trienoic acid); α-Parinaric acid (9E,11Z,13Z,15E-octadeca-9,11,13,15-tetraenoic acid); β-Parinaric acid (all trans-octadeca-9,11,13,15-tretraenoic acid); Bosseopentaenoic acid (5Z,8Z,10E,12E,14Z-eicosanoic acid); Pinolenic acid ((5Z,9Z,12Z)-octadeca-5,9,12-trienoic acid); vitamin E (α-Tocopherol, γ-Tocopherol), Podocarpic acid ((5Z,11Z,14Z)-eicosa-5,11,14-trienoic acid) and a plasmalogen.

17. The composition according to claim 10 wherein the flavan-3-ol is (−)-Epicatechin, the flavonoid is Quercetin and the fatty acid is EPA ethyl ester.

18. The composition according to claim 17 wherein the composition comprises 10-250 mg epicatechin and 10-250 mg quercetin suspended in 0.1-1 ml of eicosapentaenoic acid ethyl ester.

19. (canceled)

20. (canceled)

21. (canceled)

22. A method for treating, prophylactically treating, preventing or reducing the severity of cisplatin-induced hearing loss comprising administering to an individual in need of such treatment an effective amount of a composition comprising a flavan-3-ol, a flavonoid and a fatty acid.
